(19) United States
(12) Patent Application Publication
(10) Pub. No.: US 2026/0357353 A1
(43) Pub. Date: Nov. 12, 2026

(54) MTAP HOMOZYGOUS DELETION AND CCNE1 AMPLIFICATION IN OVARIAN SEROUS CARCINOMAS

(71) Applicant: Foundation Medicine, Inc., Boston, MA (US)

(72) Inventors: Jessica K. LEE, Somerville, MA (US); Alexa B. SCHROCK, Okemos, MI (US); Natalie DANZIGER, Cambridge, MA (US); Candice Francheska TAMBAOAN, Boston, MA (US)

(73) Assignee: Foundation Medicine, Inc., Boston, MA (US)

(21) Appl. No.: 19/548,622

(22) Filed: Feb. 24, 2026

Related U.S. Application Data
(60) Provisional application No. 63/802,198, filed on May 8, 2025.
(60) Provisional application No. 63/988,787, filed on Feb. 23, 2026.

(51) Int. Cl. C12Q 1/6886

(52) U.S. Cl. CPC C12Q 1/6886; C12Q 2600/156

(57) ABSTRACT

The present disclosure provides methods for detecting MTAP deletions (e.g., MTAP loss) as biomarkers for treatment of cancer, wherein the treatment may comprise an epigenetic modifier. The disclosure also provides methods for evaluating, identifying, assessing, and/or treating an individual having cancer with and without MTAP deletions.

### CROSS-REFERENCE TO RELATED APPLICATIONS

This application claims the priority benefit of U.S. Provisional Patent Application 63/802,198 filed May 8, 2025, and U.S. Provisional Patent Application 63/988,787, filed Feb. 23, 2026, the entire contents of each of which are incorporated herein by reference.

### FIELD

The present disclosure relates to the interaction of MTAP deletions (e.g., MTAP loss) and CCNE1 amplification as biomarkers for cancer (e.g., ovarian cancer), methods of detecting MTAP deletions and CCNE1 amplifications in samples, as well as methods of diagnosis, assessment, and treatment of cancer (e.g., ovarian cancer).

### BACKGROUND

Cancer represents the phenotypic end-point of multiple genetic lesions that endow cells with a full range of biological properties required for tumorigenesis. Indeed, a hallmark genomic feature of many cancers is the presence of numerous complex chromosome structural aberrations, including translocations, intra-chromosomal inversions, point mutations, deletions, gene copy number changes, gene expression level changes, gene fusions, and germline mutations, among others.

Methylthioadenosine phosphorylase (MTAP) alterations (including homozygous MTAP deletions, MTAP loss, deletion of MTAP exons) are emerging as biomarkers in trials evaluating PRMT5 inhibitors. Cyclin E1, encoded by the CCNE1 gene, plays an important role in cell cycle regulation and overexpression may promote unchecked cell proliferation. Early phase trials assessing WEE1, CDK2 or PKMYT1 inhibitors have shown promising antitumor activity in patients with advanced solid tumors harboring CCNE1 amp.

Accordingly, there is a need in the art for identifying novel genetic lesions associated with cancer, such as genetic lesions involving MTAP and CCNE1 The relationship between MTAP loss and CCNE1 amplifications in cancers, such as ovarian cancer was not known. Thus, a better understanding of MTAP loss and CCNE1 amplifications loss in cancer, including ovarian cancer, can inform treatment options and treatment success with WEE1, CDK2 or PKMYT1 inhibitors and/or PRMT5 inhibitors. The understanding of these genetic lesions and the relationship between them can be an effective approach to develop compositions, methods and assays for evaluating and treating cancer patients.

### BRIEF SUMMARY

Provided herein are methods of identifying treatments and treating individuals with cancer (e.g., ovarian cancer) based on knowledge of MTAP deletions and CCNE1 amplification (CCNE1-amp) status in a sample. The presence of an MTAP deletion and a CCNE1-amp negative status indicates the individual may respond to an epigenetic modifier (e.g., a PRMT5 inhibitor). The presence of an MTAP deletion and a CCNE1-amp positive status indicates the individual may respond to a combination treatment comprising an epigenetic modifier (e.g., a PRMT5 inhibitor). The combination treatment may also comprise a Wee1 inhibitor, a CDK2 inhibitor, or a PKMYT1 inhibitor. The absence of an MTAP deletion and a CCNE1-amp positive status indicates the individual may respond to a Wee1 inhibitor, a CDK2 inhibitor, or a PKMYT1 inhibitor rather than an epigenetic modifier (e.g., a PRMT5 inhibitor). The absence of an MTAP deletion and a CCNE1-amp negative status indicates the individual may not respond to a Wee1 inhibitor, a CDK2 inhibitor, a PKMYT1 inhibitor or an epigenetic modifier (e.g., a PRMT5 inhibitor). In some aspects, the individual may benefit from a platinum-based chemotherapeutic agent.

Provided herein are methods of identifying treatments and treating individuals with cancer (e.g., ovarian cancer) based on knowledge of MTAP deletions in a sample. The presence of an MTAP deletion indicates the individual may respond to an epigenetic modifier. The absence of an MTAP deletion indicates the individual may respond to an epigenetic modifier. In some aspects, the individual may benefit from a platinum-based chemotherapeutic agent.

Provided herein are methods of identifying an individual having cancer (e.g., ovarian cancer) that may benefit from a treatment with an epigenetic modifier (e.g., PRMT5 inhibitors), said method comprising: acquiring knowledge of a presence of a MTAP deletion in a sample obtained from the individual; acquiring knowledge of a CCNE1 amplification (CCNE1-amp) negative status of the sample; and identifying the individual for the treatment with the epigenetic modifier (e.g., PRMT5 inhibitors) based on the presence of a MTAP deletion in the sample and CCNE1-amp negative status of the sample.

Provided herein are methods of identifying an individual having cancer that may benefit from a treatment with an epigenetic modifier (e.g., a PRMT5 inhibitor), said method comprising: acquiring knowledge of a presence of a MTAP deletion in a sample obtained from the individual; and identifying the individual for the treatment with the epigenetic modifierbased on the presence of a MTAP deletion in the sample.

Provided herein are methods of selecting a therapy for an individual having cancer (e.g., ovarian cancer), said method comprising: acquiring knowledge of a presence of a MTAP deletion in a sample obtained from the individual; acquiring knowledge of a CCNE1 amplification (CCNE1-amp) negative status of the sample; and selecting a treatment comprising an epigenetic modifier (e.g., PRMT5 inhibitors) based on the presence of a MTAP deletion in the sample and CCNE1-amp negative status of the sample.

Provided herein are methods of selecting a therapy for an individual having cancer, said method comprising: acquiring knowledge of a presence of a MTAP deletion in a sample obtained from the individual; and selecting a treatment comprising an epigenetic modifier based on the presence of a MTAP deletion in the sample.

Provided herein are methods of identifying one or more treatment options for an individual having cancer (e.g., ovarian cancer), said method comprising: acquiring knowledge of a presence of a MTAP deletion in a sample obtained from the individual; acquiring knowledge of a CCNE1 amplification (CCNE1-amp) negative status of the sample; and generating a report comprising one or more treatment options identified for the individual based at least in part on the presence of a MTAP deletion in the sample and CCNE1-amp negative status of the sample, wherein the one or more treatment options comprise an epigenetic modifier (e.g., PRMT5 inhibitors).

Provided herein are methods of identifying one or more treatment options for an individual having cancer, said method comprising: acquiring knowledge of a presence of a MTAP deletion in a sample obtained from the individual; and generating a report comprising one or more treatment options identified for the individual based at least in part on the presence of a MTAP deletion in the sample, wherein the one or more treatment options comprise an epigenetic modifier.

Provided herein are methods of treating an individual having cancer (e.g., ovarian cancer), said method comprising: acquiring knowledge of a presence of a MTAP deletion in a sample obtained from the individual; acquiring knowledge of a CCNE1 amplification (CCNE1-amp) negative status of the sample; and administering to the individual treatment comprising an epigenetic modifier (e.g., PRMT5 inhibitors) based on the presence of a MTAP deletion in the sample and CCNE1-amp negative status of the sample.

Provided herein are methods of treating an individual having cancer, said method comprising: acquiring knowledge of a presence of a MTAP deletion in a sample obtained from the individual; and administering to the individual treatment comprising a an epigenetic modifier based on the presence of a MTAP deletion in the sample.

In some aspects, the CCNE1-amp negative status is based on CCNE1 copy number normalized by a ploidy of the sample. In some aspects, the CCNE1-amp negative status samples have a CCNE1 copy number 0 above or below the ploidy of the sample.

Provided herein are methods of identifying an individual having cancer (e.g., ovarian cancer) that may benefit from a combination treatment comprising an epigenetic modifier (e.g., PRMT5 inhibitors), said method comprising: acquiring knowledge of a presence of a MTAP deletion in a sample obtained from the individual; acquiring knowledge of a CCNE1 amplification (CCNE1-amp) positive status of the sample; and identifying the individual for the combination treatment comprising the epigenetic modifier (e.g., PRMT5 inhibitors) based on the presence of a MTAP deletion in the sample and CCNE1-amp positive status of the sample.

Provided herein are methods of selecting a therapy for an individual having cancer (e.g., ovarian cancer), said method comprising: acquiring knowledge of a presence of a MTAP deletion in a sample obtained from the individual; acquiring knowledge of a CCNE1 amplification (CCNE1-amp) positive status of the sample; and selecting a combination treatment comprising an epigenetic modifier (e.g., PRMT5 inhibitors) based on the presence of a MTAP deletion in the sample and CCNE1-amp positive status of the sample.

Provided herein are methods of identifying one or more treatment options for an individual having cancer (e.g., ovarian cancer), said method comprising: acquiring knowledge of a presence of a MTAP deletion in a sample obtained from the individual; acquiring knowledge of a CCNE1 amplification (CCNE1-amp) positive status of the sample; and generating a report comprising one or more combination treatment options identified for the individual based at least in part on the presence of a MTAP deletion in the sample and CCNE1-amp positive status of the sample, wherein the one or more combination treatment options comprise an epigenetic modifier (e.g., PRMT5 inhibitors).

Provided herein are methods of treating an individual having cancer (e.g., ovarian cancer), said method comprising: acquiring knowledge of a presence of a MTAP deletion in a sample obtained from the individual; acquiring knowledge of a CCNE1 amplification (CCNE1-amp) positive status of the sample; and administering to the individual combination treatment comprising an epigenetic modifier (e.g., PRMT5 inhibitors) based on the presence of a MTAP deletion in the sample and CCNE1-amp positive status of the sample.

In some aspects, the combination treatment further comprises a Wee1 inhibitor, a CDK2 inhibitor, or a PKMYT1 inhibitor.

In some aspects, the CCNE1-amp positive status is based on CCNE1 copy number compared to a ploidy of the sample. In some aspects, the CCNE1-amp positive status samples have a CCNE1 copy number 1 or more, 2 or more, or 3 or more above the ploidy of the sample.

Provided herein are methods of identifying an individual having cancer (e.g., ovarian cancer) that may not benefit from a treatment with an epigenetic modifier (e.g., PRMT5 inhibitors) or a Wee1 inhibitor, a CDK2 inhibitor, or a PKMYT1 inhibitor, said method comprising: acquiring knowledge of an absence of a MTAP deletion in a sample obtained from the individual; acquiring knowledge of a CCNE1 amplification (CCNE1-amp) negative status of the sample; and identifying the individual as an individual who may not benefit from an epigenetic modifier (e.g., PRMT5 inhibitors) or a Wee1 inhibitor, a CDK2 inhibitor, or a PKMYT1 inhibitor based on the absence of a MTAP deletion in the sample and CCNE1-amp negative status of the sample.

Provided herein are methods of selecting a therapy for an individual having cancer (e.g., ovarian cancer), said method comprising: acquiring knowledge of an absence of a MTAP deletion in a sample obtained from the individual; acquiring knowledge of a CCNE1 amplification (CCNE1-amp) negative status of the sample; and selecting a treatment other than a treatment comprising an epigenetic modifier (e.g., PRMT5 inhibitors) or a Wee1 inhibitor, a CDK2 inhibitor, or a PKMYT1 inhibitor based on the absence of a MTAP deletion in the sample and CCNE1-amp negative status of the sample.

Provided herein are methods of identifying one or more treatment options for an individual having cancer (e.g., ovarian cancer), said method comprising: acquiring knowledge of an absence of a MTAP deletion in a sample obtained from the individual; acquiring knowledge of a CCNE1 amplification (CCNE1-amp) negative status of the sample; and generating a report comprising one or more treatment options identified for the individual based at least in part on the absence of a MTAP deletion in the sample and CCNE1-amp negative status of the sample, wherein the one or more treatment options does not comprise an epigenetic modifier (e.g., PRMT5 inhibitors) or a Wee1 inhibitor, a CDK2 inhibitor, or a PKMYT1 inhibitor.

Provided herein are methods of treating an individual having cancer (e.g., ovarian cancer), said method comprising: acquiring knowledge of an absence of a MTAP deletion in a sample obtained from the individual; acquiring knowledge of a CCNE1 amplification (CCNE1-amp) negative status of the sample; and administering to the individual a treatment other than a treatment comprising an epigenetic modifier (e.g., PRMT5 inhibitors) or a Wee1 inhibitor, a CDK2 inhibitor, or a PKMYT1 inhibitor based on the absence of a MTAP deletion in the sample and CCNE1-amp negative status of the sample.

In some aspects, the individual may benefit from a platinum-based chemotherapeutic agent.

In some aspects, the CCNE1-amp negative status is based on CCNE1 copy number normalized by a ploidy of the sample. In some aspects, the CCNE1-amp negative status samples have a CCNE 1 copy number 0 above or below the ploidy of the sample.

Provided herein are methods of identifying an individual having cancer (e.g., ovarian cancer) that may not benefit from a treatment with an epigenetic modifier (e.g., PRMT5 inhibitors), said method comprising: acquiring knowledge of an absence of a MTAP deletion in a sample obtained from the individual: acquiring knowledge of a CCNE1-amp positive status of the sample; and identifying the individual as an individual who may not benefit from the treatment with the epigenetic modifier (e.g., PRMT5 inhibitors) based on the absence of a MTAP deletion in the sample and CCNE1-amp positive status of the sample.

Provided herein are methods of selecting a therapy for an individual having cancer (e.g., ovarian cancer), said method comprising: acquiring knowledge of an absence of a MTAP deletion in a sample obtained from the individual: acquiring knowledge of a CCNE1-amp positive status of the sample; and selecting a treatment other than a treatment comprising an epigenetic modifier (e.g., PRMT5 inhibitors) based on the absence of a MTAP deletion in the sample and CCNE1-amp positive status of the sample.

Provided herein are methods of identifying one or more treatment options for an individual having cancer (e.g., ovarian cancer), said method comprising: acquiring knowledge of an absence of a MTAP deletion in a sample obtained from the individual: acquiring knowledge of a CCNE1-amp positive status of the sample; and generating a report comprising one or more treatment options identified for the individual based at least in part on the absence of a MTAP deletion in the sample and CCNE1-amp positive status of the sample, wherein the one or more treatment options does not comprise an epigenetic modifier (e.g., PRMT5 inhibitors).

Provided herein are methods of treating an individual having cancer (e.g., ovarian cancer), said method comprising: acquiring knowledge of an absence of a MTAP deletion in a sample obtained from the individual: acquiring knowledge of a CCNE1-amp positive status of the sample; and administering to the individual a treatment other than a treatment comprising an epigenetic modifier (e.g., PRMT5 inhibitors) based on the absence of a MTAP deletion in the sample and CCNE1-amp positive status of the sample.

In some aspects, the individual may benefit from a treatment comprising a Wee1 inhibitor, a CDK2 inhibitor, or a PKMYT1 inhibitor. In some aspects, the CCNE1-amp positive status is based on CCNE1 copy number compared to a ploidy of the sample. In some aspects, CCNE1-amp positive status samples have a CCNE1 copy number 1 or more, 2 or more, or 3 or more above the ploidy of the sample.

In some aspects, the epigenetic modifier is a PRMT inhibitor and/or a PRMT5 inhibitor.

In some aspects, the cancer is ovarian cancer.

In some aspects, the ovarian cancer is ovarian serous carcinoma.

In some aspects, acquiring knowledge of the presence or absence of MTAP deletion comprises detecting the presence or absence of the MTAP deletion in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

In some aspects, the MTAP deletion is a homozygous deletion. In some aspects, the MTAP deletion comprises a homozygous deletion of the full MTAP gene, a homozygous deletion of MTAP exons 1-8, a homozygous deletion of MTAP exons 5-8, a homozygous deletion of MTAP exons 2-8, a homozygous deletion of MTAP exons 6-8, a homozygous deletion of MTAP exons 7-8, a homozygous deletion of MTAP exon 8, a homozygous deletion of MTAP exons 3-8 or a homozygous deletion of MTAP exons 4-8.

In some aspects, acquiring knowledge of the CCNE1-amp negative status comprises determining a CCNE1-amp status in the sample. In some aspects, acquiring knowledge of the CCNE1-amp positive status comprises determining a CCNE1-amp status in the sample. In some aspects, a CCNE1-amp status in the sample comprises calculating a copy number of CCNE1 in the sample and a ploidy of the sample. In some aspects, the copy number of CCNE1 in the sample and a ploidy of the sample is determined in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

In some aspects, determining a CCNE1-amp negative status of the sample increases a probability of a detecting a presence of a MTAP deletion in the sample. In some aspects, determining a CCNE1-amp positive status of the sample increases a probability of detecting an absence of the MTAP deletion in the sample. In some aspects, the sample comprises a tissue biopsy sample or a liquid biopsy. In some aspects, the sample is a tissue biopsy and comprises a tumor biopsy.

In some aspects, the sample is a liquid biopsy sample and comprises blood, serum, plasma, cerebrospinal fluid, sputum, stool, urine, or saliva. In some aspects, the sample comprises cells and/or nucleic acids from the cancer (e.g., ovarian cancer). In some aspects, the sample comprises mRNA, DNA, circulating tumor DNA (ctDNA), cell-free DNA, cell-free RNA from the cancer (e.g., ovarian cancer), or any combination thereof. In some aspects, the sample is a liquid biopsy sample and comprises circulating tumor cells (CTCs). In some aspects, the sample is a liquid biopsy sample and comprises cell-free DNA (cfDNA), circulating tumor DNA (ctDNA), or any combination thereof.

In some aspects, the methods further comprise obtaining the sample from the individual. In some aspects, the methods further comprise obtaining the liquid biopsy sample from the individual. In some aspects, the methods further comprise obtaining the tissue biopsy sample from the individual.

In some aspects, the sequencing comprises a massively parallel sequencing (MPS) technique, whole genome sequencing (WGS), whole exome sequencing, targeted sequencing, direct sequencing, or a Sanger sequencing technique; an optionally wherein the massively parallel sequencing (MPS) technique comprises next-generation sequencing.

In some aspects, the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample.

In some aspects, the one or more bait molecules comprise one or more nucleic acid molecules, each comprising a region that is complementary to a region of a captured nucleic acid molecule. In some aspects, the one or more bait molecules each comprise a capture moiety. In some aspects, the capture moiety is biotin.

In some aspects, the individual is a patient. In some aspects, the individual is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

FIG. 1 demonstrates the enrichment of genomic alterations in ovarian cancer samples with no CCNE1 gain or a CCNE1 AmpRatio greater than or equal to 3. The points are colored by alteration type (CN: copy number, SV: short variant such as a point mutation or indel, RE: rearrangement) and are sized according to overall prevalence.

FIG. 2 demonstrates the enrichment of genomic alterations in ovarian cancer samples with varying CCNE1 AmpRatio (CCNE1 AmpRatio as a continuous variable). The points are colored by alteration type (CN: copy number, SV: structural variant, RE: rearrangement) and are sized according to overall prevalence.

FIG. 3 demonstrates observed MTAP exon deletion genotypes for serous ovarian cancer carcinoma samples, sorted by prevalence from most prevalent at the top to least prevalent at the bottom.

### DETAILED DESCRIPTION

The present disclosure is based, at least in part, on the discovery of the relationship between MTAP deletion and CCNE1 amplification (CCNE1-amp) status in cancer, e.g., ovarian cancer or ovarian serous carcinoma. The relationship informs how treatments potentially indicated for cancers with an MTAP deletion, such as Wee1, CDK2 or PKMYT1 inhibitors and treatments for cancers with CCNE1 amplification, such as treatments that target PRMT5 can be used to effectively and efficiently treat patients with cancer, e.g., ovarian cancer or ovarian serous carcinoma. It was identified that MTAP deletions and CCNE1 amplification are often mutually exclusive which may inform therapeutic strategies for patients based on these biomarkers. In some aspect, the MTAP deletion is a homozygous deletion of MTAP and may be referred to as MTAP loss.

The methods provided herein can be used to identifying individuals with cancer (e.g., ovarian cancer) who may benefit from treatment with an epigenetic modifier (e.g., aPRMT5 inhibitor), treatment comprising Wee1, CDK2 and PKMYT1 inhibitors, either treatment or both treatments. The methods may be used to identifying an individual with cancer (e.g., ovarian cancer) who may not benefit from treatment with the epigenetic modifier (e.g., a PRMT5 inhibitor), treatment comprising Wee1, CDK2 and PKMYT1 inhibitors, either treatment or both treatments. In some aspects, the individual with cancer may benefit from treatment with a platinum based chemotherapeutic agent.

The methods provided herein can be used to identify individuals with cancer who may benefit from treatment with an epigenetic modifier based on the presence or absence of an MTAP deletion in a sample from the individual. In some embodiments, the methods are for treating or delaying progression of cancer. In some embodiments, presence of an MTAP deletion indicates the individual will benefit from treatment with the epigenetic modifier. In some embodiments, the cancer is ovarian cancer. In some embodiments, the epigenetic modifier is a PRMT inhibitor or a PRMT5 inhibitor.

In other aspects, provided herein are methods of treating or delaying progression of cancer, e.g., ovarian cancer or ovarian serous carcinoma. In other aspects, provided herein are methods of identifying one or more treatment options for an individual having cancer, e.g., ovarian cancer or ovarian serous carcinoma. In other aspects, provided herein are methods of selecting treatment for an individual having cancer, e.g., ovarian cancer or ovarian serous carcinoma. In other aspects, provided herein are methods of screening an individual havingcancer, suspected of having cancer, being tested for cancer, being treated for cancer, or being tested for a susceptibility to cancer, e.g., ovarian cancer or ovarian serous carcinoma. In other aspects, provided herein are methods of identifying an individual having cancer who may benefit from a treatment comprising an anti-cancer therapy, such as an epigenetic modifier (e.g., PRMT5 inhibitors) and/or Wee1, CDK2, or PKMYT1 inhibitors. In other aspects, provided herein are methods of selecting a therapy for an individual having cancer. In some embodiments, the cancer comprises a MTAP deletion and/or a CCNE1 amplification positive status.

In some embodiments, the methods comprise acquiring knowledge of the presence or absence of a MTAP deletion in a sample from an individual. In some embodiments, the methods comprise acquiring knowledge of the CCNE1-amp status in a sample from an individual. In some embodiments, the methods comprise generating a report comprising one or more treatment options for the individual. In some embodiments, the methods comprise administering to an individual having cancer an effective amount of a treatment comprising an anti-cancer therapy. In some embodiments, the cancer is ovarian cancer. In some embodiments, the cancer is an ovarian serous carcinoma. In some embodiments, the anti-cancer therapy is an anti-cancer therapy described herein, such as an epigenetic modifier, aPRMT5 inhibitor and/or a Wee1, CDK3, or PKMYT1 inhibitor.

In some embodiments, the individual is a human. In some embodiments, the individual is a patient.

In other aspects, provided herein are anti-cancer therapies for use in a method of treating or delaying progression of cancer, (e.g., ovarian cancer) such as a cancer comprising a MTAP deletion. The cancer may be CCNE1-amp positive status or CCNE1-amp negative status. In other aspects, provided herein are anti-cancer therapies for use in the manufacture of a medicament for treating or delaying progression of cancer, such as a cancer comprising a MTAP deletion.

In other aspects, provided herein are in vitro uses of one or more oligonucleotides, probes, baits or antibodies for detecting or isolating MTAP and/or CCNE1 nucleic acid molecule or polypeptide described herein. In other aspects, provided herein are kits comprising one or more oligonucleotides, probes, baits or antibodies for detecting or isolating MTAP and/or CCNE1 nucleic acid molecule or polypeptide described herein.

### Definitions

Before describing the invention in detail, it is to be understood that this invention is not limited to particular compositions or biological systems. It is also to be understood that the terminology used herein is for the purpose of describing particular embodiments only, and is not intended to be limiting.

Unless otherwise defined, all of the technical terms used herein have the same meaning as commonly understood by one of ordinary skill in the art in the field to which this disclosure belongs.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

The term “isolated” in the context of a nucleic acid molecule or a polypeptide refers to a nucleic acid molecule or polypeptide being separated from other nucleic acid molecules or polypeptides that are present in the natural source of the nucleic acid molecule or polypeptide. In some certain embodiments, the isolated nucleic acid molecule or polypeptide is free of or substantially free of other cellular material or culture medium when produced by recombinant techniques, or free of or substantially free of chemical precursors or other chemicals when chemically synthesized.

As used herein, the term “configured to hybridize to” indicates that a nucleic acid molecule has a nucleotide sequence with sufficient length and sequence complementarity to the nucleotide sequence of a target nucleic acid to allow the nucleic acid molecule to hybridize to the target nucleic acid, e.g., with a Tₘ of at least 65 °C in an aqueous solution of 1× SCC (150 mM sodium chloride and 15 mM trisodium citrate) and 0.1% SDS. Other hybridization conditions may be used when hybridizing a nucleic acid molecule to a target nucleic acid molecule, for example in the context of a described method.

“Percent (%) sequence identity” with respect to a reference polypeptide or polynucleotide sequence is defined as the percentage of amino acid residues or nucleotides in a sequence that are identical to the amino acid residues or nucleotides in the reference polypeptide or polynucleotide sequence, after aligning the sequences and introducing gaps, if necessary, to achieve the maximum percent sequence identity, and not considering any conservative substitutions as part of the sequence identity.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the individual is human patient, e.g., a human patient having a cancer described herein.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Likely to” or “increased likelihood,” as used herein, refer to an increased probability that an event, item, object, thing or person will occur. Thus, in one example, an individual that is likely to respond to treatment with an anti-cancer therapy, e.g., an anti-cancer therapy provided herein, alone or in combination, has an increased probability of responding to treatment with the anti-cancer therapy alone or in combination, relative to a reference individual or group of individuals. “Unlikely to” refers to a decreased probability that an event, item, object, thing or person will occur relative to a reference individual or group of individuals. Thus, an individual that is unlikely to respond to treatment with an anti-cancer therapy, e.g., an anti-cancer therapy provided herein, alone or in combination, has a decreased probability of responding to treatment with the anti-cancer therapy, alone or in combination, relative to a reference individual or group of individuals.

“Sample,” as used herein, refers to a biological sample obtained or derived from a source of interest, as described herein.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

The section headings used herein are for organizational purposes only and are not to be construed as limiting the subject matter described.

Finally, it is to be understood that features and preferences described in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein. Further, sectional headings are provided for organizational purposes and are not to be considered limiting.

### Methods of Acquiring Knowledge of a MTAP Deletion

In some aspects, provided herein are methods comprising acquiring knowledge of the presence or absence of a MTAP deletion in a sample collected from an individual with cancer (e.g., ovarian cancer). Acquiring knowledge of the presence or absence of a MTAP deletion may comprise detected MTAP, including MTAP nucleic acid molecules and MTAP polypeptide molecules. An MTAP deletion may comprise a homozygous deletion of the full MTAP gene. In some aspects, the MTAP deletion may comprise a homozygous deletion of at least a subset of exons from the MTAP gene. In some aspects, the MTAP deletion may comprise a deletion of any combination of MTAP exons. In some aspects, the MTAP deletion may comprise a homozygous deletion of exons 1-8. In some aspects, the MTAP deletion may comprise a homozygous deletion of exons 5-8. In some aspects, the MTAP deletion may comprise a homozygous deletion of exons 2-8. In some aspects, the MTAP deletion may comprise a homozygous deletion of exons 6-8. In some aspects, the MTAP deletion may comprise a homozygous deletion of exons 7-8. In some aspects, the MTAP deletion may comprise a homozygous deletion of exon 8. In some aspects, the MTAP deletion may comprise a homozygous deletion of exons 3-8. In some aspects, the MTAP deletion may comprise a homozygous deletion of exons 4-8. In some aspects, the MTAP deletion may result in loss of function of MTAP.

In some aspects, the presence of an MTAP deletion in a sample from an individual with cancer, e.g., ovarian cancer or ovarian serous carcinoma, may be indicative that the sample is less likely to have a CCNE1-amp positive status.

In some aspects, acquiring knowledge of the presence or absence of a MTAP deletion comprises detecting the presence or absence of an MTAP deletion in a sample from an individual with cancer, e.g., ovarian cancer or ovarian serous carcinoma.

In some aspects, provided herein are methods of detecting the presence of a MTAP deletion as described herein, e.g., in a sample. In some embodiments, the sample is obtained from an individual, such as an individual having cancer. In some embodiments, the sample is obtained from an individual, such as an individual having ovarian cancer, e.g., ovarian serous carcinoma. In some aspects, detecting MTAP nucleic acids and/or MTAP polypeptides indicates the absence of an MTAP deletion in the sample. In some embodiments, the methods of detecting the presence of a MTAP nucleic acid molecule described herein comprise selectively enriching for one or more nucleic acids comprising MTAP nucleotide sequences to produce an enriched sample, e.g., using a reagent known in the art or provided herein, such as a bait, probe, or oligonucleotide described herein. In some embodiments, the methods of detecting the presence of a MTAP polypeptide described herein comprise selectively enriching for one or more polypeptides comprising MTAP amino acid sequences to produce an enriched sample, e.g., using a reagent known in the art or provided herein, such as an antibody described herein.

In some aspect, the methods described herein, including but not limited to, a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing can be used to detect the presence or absence of a MTAP deletion.

The knowledge of the presence of a MTAP deletion may be used according to the methods described herein in a variety of applications such as, but not limited to, identifying an individual having cancer (e.g., ovarian cancer) who may benefit from treatment with an epigenetic modifier (e.g., a PRMT5 inhibitor), identifying one or more treatment options for the individual having cancer (e.g., ovarian cancer) wherein the one or more treatment options comprise an epigenetic modifier (e.g., a PTRM5 inhibitor), or methods of treating an individual having cancer (e.g., ovarian cancer) with an epigenetic modifier (e.g., a PRMT5 inhibitor). In some aspects, the knowledge of CCNE1 amplification status of the sample may be used and may inform treatment selection or treatment with a combination treatment comprising an epigenetic modifier (e.g., a PRMT5 inhibitor). The combination treatment may comprise a Wee1 inhibitor, a CDK2 inhibitor, or a PKMYT1 inhibitor.

The knowledge of the absence of a MTAP deletion may be used according to the methods described herein in a variety of applications such as, but not limited to, identifying an individual having cancer (e.g., ovarian cancer) who may not benefit from treatment with an epigenetic modifier (e.g., a PRMT5 inhibitor), identifying one or more treatment options for the individual having cancer (e.g., ovarian cancer) wherein the one or more treatment options does not comprise an epigenetic modifier (e.g., a PTRM5 inhibitor), or methods of treating an individual having cancer (e.g., ovarian cancer) with a treatment other than an epigenetic modifier (e.g., a PRMT5 inhibitor). In some aspects, the knowledge of CCNE1 amplification status of the sample may be used and may inform treatment selection or treatment with a Wee1 inhibitor, a CDK2 inhibitor, or an epigenetic modifier (e.g., an PKMYT1 inhibitor). In some aspects, a treatment option of other than an epigenetic modifier (e.g., a PTRMT5 inhibitor) may comprise a platinum based chemotherapeutic agent and/or a treatment comprising a Wee1 inhibitor, a CDK2 inhibitor, or a PKMYT1 inhibitor.

The knowledge of the presence of a MTAP deletion may be used according to the methods described herein in a variety of applications such as, but not limited to, identifying an individual having cancer who may benefit from treatment with an epigenetic modifier, identifying one or more treatment options for the individual having cancer wherein the one or more treatment options comprise an epigenetic modifier, or methods of treating an individual having cancer with an epigenetic modifier. In some embodiments, the cancer is ovarian cancer. In some embodiments, the epigenetic modifier is a PRMT inhibitor or a PRMT5 inhibitor.

The knowledge of the absence of a MTAP deletion may be used according to the methods described herein in a variety of applications such as, but not limited to, identifying an individual having cancer who may not benefit from treatment with an epigenetic modifier, identifying one or more treatment options for the individual having cancer, wherein the one or more treatment options does not comprise an epigenetic modifier, or methods of treating an individual having cancer with a treatment other than an epigenetic modifier. In some embodiments, the cancer is ovarian cancer. In some embodiments, the epigenetic modifier is a PRMT inhibitor or a PRMT5 inhibitor.

### Methods of Acquiring Knowledge of CCNE1 Amplification Status

In some aspects, provided herein are methods comprising acquiring knowledge of a CCNE1 amplification (CCNE1-amp) positive status in a sample from an individual with cancer (e.g., ovarian cancer). A CCNE1-amp positive status may be defined based on the CCN1 copy number in the sample normalized by the ploidy of the sample. In some aspect, a sample may be CCNE1-amp positive if the amplification ratio (AmpRatio), defined as the copy number of CCNE1 in the sample normalized by the ploidy of the sample is greater than or equal to 3. In some aspects, a sample may be CCNE1-amp positive if the CCNE1 copy number is greater than the ploidy of the sample. For example, of the CCNE 1 copy number is 1 above, 2, about or 3 above the ploidy of the sample.

In some aspects, provided herein are methods comprising acquiring knowledge of a CCNE1 amplification (CCNE1-amp) negative status in a sample from an individual with cancer (e.g., ovarian cancer). A CCNE1-amp negative status may be defined based on the CCN1 copy number in the sample normalized by the ploidy of the sample. In some aspect, a sample may be CCNE1-amp negative if the amplification ratio (AmpRatio), defined as the copy number of CCNE 1 in the sample normalized by the ploidy of the sample is 0. In some aspects, a sample may be CCNE1-amp negative if the CCNE1 copy number is the same or less than the ploidy of the sample. In some aspects, a sample may be CCNE1-amp negative if the CCNE1 copy number is 0 above, or below the ploidy the sample. For example, of the CCNE1 copy number is 0 below, 1 below, 2 below, or 3 below the ploidy of the sample.

In some aspects, the CCNE1-amp status of a sample from an individual with cancer, e.g., an ovarian cancer or ovarian serous carcinoma, may be indicative of the MTAP loss status of the sample. CCNE1-amp positive status may be indicative of the absence of a MTAP deletion. CCNE1-amp negative status may be indicative of the presence of a MTAP deletion.

In some aspects, provided herein are methods of determining a CCNE1-amp status of a sample from an individual with cancer, e.g., an ovarian cancer, ovarian serous carcinoma. Determining a CCNE1-amp status may comprise determining a CCNE1 AmpRatio for the sample. In some aspects, determining a CCNE1-amp status and/or CCNE1 AmpRatio may comprise calculating a copy number of CCNE1 and the ploidy of the sample. The ploidy of the sample may be the average copy number of all of the subgenomic intervals sequenced from the sample. In some aspects, the copy number of CCNE1 in the sample and the ploidy of the sample may be determined un the sample by one or more of copy number of CCNE1 in the sample and a ploidy of the sample is determined in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some aspects, the methods may comprise selectively enriching for CCNE1 nucleotides to produce an enriched sample, e.g., using a reagent known in the art or provided herein, such as a bait, probe, or oligonucleotide described herein.

In some aspects, the methods described herein comprise determining a CCNE1 AmpRatio for a sample. The methods may comprise calculating a copy number of CCNE1 and the ploidy of the sample. In some aspects, methods known in the art for calculating copy number of a gene and sample ploidy may be used. In some aspects, the methods for calculating copy number from a sample may comprise the methods described in U.S. patent application Ser. No. 18/698,746 (US Patent publication US2024-0404626-A1, PCT Publication WO2023060236A1), U.S. patent application Ser. No. 18/999,895 (PCT publication WO2024006744A2), and U.S. patent application Ser. No. 18/698,747 (U.S. Patent Publication US-2024-0412812-A1, PCT Publication WO2023060261A1), each of which are hereby incorporated by reference in their entirety.

The knowledge of a CCNE1-amp positive or negative status may be used according to the methods described herein in a variety of applications such as, but not limited to, identifying an individual having cancer (e.g., ovarian cancer) who may benefit from treatment with an epigenetic modifier (e.g., a PRMT5 inhibitor), identifying an individual having ovarian cancer who may benefit from treatment with a Wee1 inhibitor, a CDK2 inhibitor, or a PKMYT1 inhibitor, identifying one or more treatment options for the individual having cancer (e.g., ovarian cancer) wherein the one or more treatment options comprise an epigenetic modifier (e.g., a PTRM5 inhibitor) and/or a Wee1 inhibitor, a CDK2 inhibitor, or a PKMYT1 inhibitor, or methods of treating an individual having cancer (e.g., ovarian cancer) with an epigenetic modifier (e.g., a PRMT5 inhibitor) and/or a Wee1 inhibitor, a CDK2 inhibitor, or a PKMYT1 inhibitor. In some aspects, the knowledge of the presence or absence of a MTAP deletion in the sample may be used and may inform treatment selection or treatment with a combination treatment comprising an epigenetic modifier (e.g., a PRMT5 inhibitor) or a treatment other than a Wee1 inhibitor, a CDK2 inhibitor, or a PKMYT1 inhibitor or an epigenetic modifier (e.g., a PRMT5 inhibitor) (e.g., platinum based chemotherapeutic agent). The combination treatment may comprise a Wee1 inhibitor, a CDK2 inhibitor, or a PKMYT1 inhibitor.

### Detection of MTAP and CCNE1 Nucleic Acids and Polypeptides

Provided herein are methods that can be used to detect a MTAP deletion (e.g., MTAP loss). The MTAP deletion may be a homozygous deletion of MTAP. In some aspects, detecting MTAP loss. Also provided herein are methods that can be used to detect CCNE1 in order to determine a CCNE1-amp status.

In some embodiments, a MTAP deletion may be detected in a sample (e.g., liquid biopsy sample or tissue sample) by one or more of as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety.

In some embodiments, CCNE1 nucleic acids may be detected in a sample (e.g., liquid biopsy sample or tissue sample) by one or more of as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety.

In some aspects, provided herein are reagents that can be used for detecting the presence or absence of MTAP and/or CCNE1 nucleic acid molecules of the disclosure or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises a deletion of MTAP, or multiple copies of CCNE1 described herein or a fragment or portion thereof.

### In Situ Hybridization Methods

In some embodiments, a in situ hybridization methods may be used to detect the presence or absence of MTAP and/or CCNE1 nucleic acid molecules of the disclosure. The nucleic acids may be detected using an in situ hybridization method, such as a fluorescence in situ hybridization (FISH) method.

In some embodiments, FISH analysis is used to identify the chromosomal rearrangement resulting in the mutations as described herein. In some embodiments, FISH analysis is used to identify an RNA molecule comprising a MTAP and/or CCNE1 nucleic acid. Methods for performing FISH are known in the art and can be used in nearly any type of tissue. In FISH analysis, nucleic acid probes which are detectably labeled, e.g., fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA comprising MTAP and/or CCNE1 as provided herein.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a MTAP deletion and/or CCNE1-amp positive status as described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

### Array-based Methods

In some embodiments, an array based methods may be used to detect the presence or absence of MTAP and/or CCNE1 nucleic acid molecules of the disclosure. An array-based method, such as array-based comparative genomic hybridization (CGH) methods may be used. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

In some embodiments, the capture nucleic acid molecule is configured to hybridize to a fragment of the MTAP and/or CCNE1 nucleic acid molecule. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 n nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, a bait provided herein comprises a DNA, RNA, or a DNA/RN molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

### Amplification-based Methods

In some embodiments, amplification-based methods may be used to detect the presence or absence of MTAP and/or CCNE1 nucleic acid molecules of the disclosure. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of a MTAP deletion of the disclosure in the sample can be determined based on the presence or absence of an amplification product. Quantitative amplification methods are also known in the art and may be used according to the methods provided herein, such as for determining copy number of CCNE1 or the ploidy of a sample. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

### Sequencing

In some embodiments, a MTAP deletion and/or a CCNE1-amp status may be detected using a sequencing method. Any method of sequencing known in the art can be used to detect a MTAP and/or CCNE1 nucleic acid molecules as provided herein. Sequencing of the MTAP and/or CCNE1 nucleic acid molecules can be used for identifying a MTAP deletion and/or CCNE1-amp status. Exemplary sequencing methods that may be used to detect a MTAP deletion and/or determine a CCNE1-amp status include those based on techniques developed by Maxam and Gilbert or Sanger. Automated sequencing procedures may also be used, e.g., including sequencing by mass spectrometry.

In some embodiments, a MTAP and/or CCNE1 nucleic acid molecule of the disclosure is detected using hybrid capture-based sequencing (hybrid capture-based NGS), e.g., using adaptor ligation-based libraries. See, e.g., Frampton, G. M. et al. (2013) Nat. Biotech. 31:1023-1031. In some embodiments, a MTAP and/or CCNE1 nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a MTAP and/or CCNE1 nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect a MTAP and/or CCNE1 nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

### Detection Reagents

In some aspects, provided herein are reagents for detecting a MTAP and/or CCNE1 nucleic acid molecule of the disclosure or a fragment thereof, e.g., according to the methods of detection provided herein. In some aspect, detecting MTAP and/or CCNE1 nucleic acid molecules can be used for detecting a MTAP deletion or determining a CCNE1-amp status of a sample. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises a MTAP and/or CCNE1 nucleic acid molecule described herein or a fragment or portion thereof.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA comprising a MTAP and/or CCNE1 sequence or a fragment thereof.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a MTAP deletion and/or a CCNE1-amp positive status described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)-or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule comprising a MTAP and/or CCNE1 nucleic acid molecule, or a fragment or portion thereof. In some embodiments, the oligonucleotide comprises a nucleotide sequence configured to hybridize to the MTAP and/or CCNE1 nucleic acid molecule of the target nucleic acid molecule. In some embodiments, the oligonucleotide comprises a nucleotide sequence configured to hybridize to a fragment or portion of the MTAP and/or CCNE1 nucleic acid molecule of the target nucleic acid molecule.

In some embodiments, the oligonucleotide comprises a nucleotide sequence corresponding to a MTAP and/or CCNE1 nucleic acid molecule provided herein. In some embodiments, the oligonucleotide comprises a nucleotide sequence corresponding to a fragment or a portion of a MTAP and/or CCNE1 nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a MTAP and/or CCNE1 nucleic acid molecule. In some embodiments, the oligonucleotide comprises a nucleotide sequence complementary to a fragment or a portion of a MTAP and/or CCNE1 nucleic acid molecule. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule comprising a MTAP and/or CCNE1 nucleic acid molecule, or a fragment thereof, e.g., using PCR. In some embodiments, the oligonucleotide may be used to sequence a nucleic acid molecule comprising a MTAP and/or CCNE1 nucleic acid molecule, or a fragment thereof. In some aspects, lack of binding of the oligonucleotide to an MTAP nucleic acid or a fragment thereof may be indicative of MTAP loss in a sample.

In some embodiments, pairs of oligonucleotides, e.g., pairs of primers, are provided herein, which are configured to hybridize to a nucleic acid molecule comprising a MTAP and/or CCNE1 nucleic acid molecule provided herein, or a fragment thereof. In some embodiments, a pair of oligonucleotides of the disclosure may be used for directing amplification of the MTAP and/or CCNE1 nucleic acid molecule, e.g., using a PCR reaction. In some aspects, lack of binding of the pair oligonucleotide to an MTAP nucleic acid or a fragment thereof may be indicative of MTAP loss in a sample.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a MTAP and/or CCNE1 nucleic acid molecule, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a MTAP deletion described herein, from a reference nucleotide sequence.

In one aspect, provided herein is a primer or primer set for amplifying a nucleic acid molecule comprising a cytogenetic abnormality such as a chromosomal inversion, deletion, translocation, duplication, or other rearrangement resulting in a MTAP deletion and/or amplification of CCNE1. In another aspect, provided herein is a primer or primer set for amplifying a nucleic acid molecule comprising a chromosomal inversion, insertion, deletion, translocation, duplication or other rearrangement resulting in a MTAP deletion and/or amplification of CCNE1.

### Protein Detection Based Methods

In some aspects, protein based detection methods may be used to detect the presence or absence of MTAP and/or CCNE1-amp status. Also provided herein are methods of detecting a MTAP and/or CCNE1 polypeptide of the disclosure, or a fragment thereof in order to detect the presence or absence of an MTAP deletion and/or the CCNE1-amp status of a sample. A MTAP and/or CCNE1 polypeptide, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some aspects, methods of detection of a MTAP and/or CCNE1 polypeptide of the disclosure, or a fragment thereof, are provided, comprising contacting a sample, e.g., a sample described herein, comprising MTAP and/or CCNE1 polypeptide described herein, with a detection reagent provided herein (e.g., an antibody of the disclosure), and determining if the MTAP and/or CCNE1 polypeptide is present in the sample.

In some embodiments, antibodies or antibody fragments that specifically bind to a MTAP and/or CCNE1 polypeptide may be used to detect MTAP deletions or determine CCNE1-amp status. The antibody may be of any suitable type of antibody or antibody fragment, including, but not limited to, a monoclonal antibody, a polyclonal antibody, a multi-specific antibody (e.g., a bispecific antibody), or an antibody fragment, so long as the antibody or antibody fragment exhibits a specific antigen binding activity (e.g., binding to a MTAP and/or CCNE1 polypeptide, or a portion thereof). In some embodiments, the antibody may be a chimeric or humanized antibody. In certain embodiments, an antibody provided herein is a human antibody.

### Methods Selecting or Identifying a Treatment

In some aspects, provided herein are methods of identifying an individual having cancer. In some aspects, provided herein are methods of identifying an individual having ovarian cancer, e.g., ovarian serous carcinoma, who may benefit from an anti-cancer therapy, e.g., an anti-cancer therapy provided herein. In some embodiments, the methods comprise acquiring knowledge of the presence or absence of a MTAP deletion. In some embodiments, the methods comprise detecting the presence or absence of a MTAP deletion. In some embodiments, the presence or absence of the MTAP deletion in the sample identifies the individual as one who may benefit from a treatment comprising an anti-cancer therapy, e.g., an anti-cancer therapy provided herein. In some embodiments, responsive to knowledge of the presence or absence of the MTAP deletion, the individual is identified as one who may benefit from a treatment comprising an anti-cancer therapy, e.g., an anti-cancer therapy provided herein. In some embodiments, the presence or absence of the MTAP deletion may be detected may be determined using any suitable method known in the art or described herein. In some embodiments, the sample is a sample described herein. In some embodiments, the sample comprises cells from the cancer (e.g., ovarian cancer) or is obtained from cells from the cancer. In some embodiments, the anti-cancer therapy comprises an epigenetic modifier. In some embodiments, the epigenetic modifier is a PRMT inhibitor or a PRMT5 inhibitor.

In some aspects, provided herein are methods of identifying an individual having cancer. In some aspects, provided herein are methods of identifying an individual having ovarian cancer, e.g., ovarian serous carcinoma, who may benefit from an anti-cancer therapy, e.g., an anti-cancer therapy provided herein. In some embodiments, the methods comprise acquiring knowledge of the presence or absence of a MTAP deletion. In some embodiments, the methods comprise acquiring knowledge of the presence or absence of a MTAP deletion and a CCNE1-amp status as provided herein in a sample obtained from the individual. In some embodiments, the methods comprise detecting the presence or absence of a MTAP deletion. In some embodiments, the methods comprise detecting the presence or absence of a MTAP deletion and/or determining a CCNE1-amp status in a sample obtained from the individual. In some embodiments, the presence or absence of the MTAP deletion and CCNE1-amp status in the sample identifies the individual as one who may benefit from a treatment comprising an anti-cancer therapy, e.g., an anti-cancer therapy provided herein. In some embodiments, responsive to knowledge of the presence or absence of the MTAP deletion and the CCNE1-amp status, the individual is identified as one who may benefit from a treatment comprising an anti-cancer therapy, e.g., an anti-cancer therapy provided herein. In some embodiments, the presence or absence of the MTAP deletion may be detected and the CCNE1-amp status may be determined using any suitable method known in the art or described herein. In some embodiments, the sample is a sample described herein. In some embodiments, the sample comprises cells from the cancer (e.g., ovarian cancer) or is obtained from cells from the cancer.

In some aspects, provided herein are methods of identifying an individual having ovarian cancer, e.g., ovarian serous carcinoma, who may benefit from treatment with a PRMT5 inhibitor. In some embodiments, the methods comprise acquiring knowledge of the presence of a MTAP deletion and a CCNE1-amp negative status as provided herein in a sample obtained from the individual. In some embodiments, the methods comprise detecting the presence of a MTAP deletion and/or determining a CCNE1-amp negative status in a sample obtained from the individual. In some embodiments, the presence of the MTAP deletion and CCNE1-amp negative status in the sample identifies the individual as one who may benefit from treatment with an epigenetic modifier (e.g., a PRMT5 inhibitor). In some embodiments, responsive to knowledge of the presence of the MTAP deletion and the CCNE1-amp negative status, the individual is identified as one who may benefit from a treatment comprising an epigenetic modifier (e.g., a PRMT5 inhibitor). In some embodiments, the presence of the MTAP deletion may be detected and the CCNE1-amp negative status may be determined using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer). In some embodiments, the epigenetic modifier is a PRMT inhibitor or a PRMT5 inhibitor.

In some aspects, provided herein are methods of identifying an individual having cancer, e.g., an ovarian cancer or ovarian serous carcinoma, who may benefit from a combination treatment comprising an epigenetic modifier (e.g., a PRMT5 inhibitor). In some embodiments, the methods comprise acquiring knowledge of the presence of a MTAP deletion and a CCNE1-amp positive status as provided herein in a sample obtained from the individual. In some embodiments, the methods comprise detecting the presence of a MTAP deletion and/or determining a CCNE1-amp positive status in a sample obtained from the individual. In some embodiments, the presence of the MTAP deletion and CCNE1-amp positive status in the sample identifies the individual as one who may benefit from a combination treatment comprising an epigenetic modifier (e.g., a PRMT5 inhibitor). In some embodiments, responsive to knowledge of the presence of the MTAP deletion and the CCNE1-amp positive status, the individual is identified as one who may benefit from a combination treatment comprising an epigenetic modifier (e.g., a PRMT5 inhibitor). In some aspects, the combination treatment further comprises a Wee1 inhibitor, a CDK2 inhibitor, or a PKMYT1 inhibitor. In some embodiments, the presence of the MTAP deletion may be detected and the CCNE1-amp positive status may be determined using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer).

In some aspects, provided herein are methods of identifying an individual having cancer, e.g., an ovarian cancer or ovarian serous carcinoma, who may not benefit from a treatment with an epigenetic modifier (e.g., a PRMT5 inhibitor) or a Wee1 inhibitor, a CDK2 inhibitor, or a PKMYT1 inhibitor. In some embodiments, the methods comprise acquiring knowledge of the absence of a MTAP deletion and a CCNE1-amp negative status as provided herein in a sample obtained from the individual. In some embodiments, the methods comprise detecting the absence of a MTAP deletion and/or determining a CCNE1-amp negative status in a sample obtained from the individual. In some embodiments, the absence of the MTAP deletion and CCNE1-amp negative status in the sample identifies the individual as one who may not benefit from a treatment with an epigenetic modifier (e.g., a PRMT5 inhibitor) or a Wee1 inhibitor, a CDK2 inhibitor, or a PKMYT1 inhibitor. In some embodiments, responsive to knowledge of the absence of the MTAP deletion and the CCNE1-amp negative status, the individual is identified as one who may not benefit from a treatment. In some aspects, the individual may benefit from a standard of care anti-cancer therapy, such as an anti-cancer therapy described herein. In some aspect, the individual may benefit from a platinum-based chemotherapeutic agent. In some embodiments, the absence of the MTAP deletion may be detected and the CCNE1-amp negative status may be determined using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer).

In some aspects, provided herein are methods of identifying an individual having cancer, e.g., an ovarian cancer or ovarian serous carcinoma, who may not benefit from a treatment with an epigenetic modifier (e.g., a PRMT5 inhibitor). In some embodiments, the methods comprise acquiring knowledge of the absence of a MTAP deletion and a CCNE1-amp positive status as provided herein in a sample obtained from the individual. In some embodiments, the methods comprise detecting the absence of a MTAP deletion and/or determining a CCNE1-amp positive status in a sample obtained from the individual. In some embodiments, the absence of the MTAP deletion and CCNE1-amp positive status in the sample identifies the individual as one who may not benefit from a treatment comprising an epigenetic modifier (e.g., a PRMT5 inhibitor). In some embodiments, responsive to knowledge of the absence of the MTAP deletion and the CCNE1-amp positive status, the individual is identified as one who may not benefit from a treatment comprising an epigenetic modifier (e.g., a PRMT5 inhibitor). In some aspects, the individual may benefit from treatment comprising a Wee1 inhibitor, a CDK2 inhibitor, or a PKMYT1 inhibitor. In some embodiments, the absence of the MTAP deletion may be detected and the CCNE1-amp positive status may be determined using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer).

Also provided herein are methods of identifying or selecting a treatment, a therapy, or one or more treatment options for an individual having a cancer, such as an ovarian cancer described herein. In some embodiments, the methods comprise obtaining knowledge of the presence or absence of a MTAP deletion and the CCNE1-amp status in a sample from the individual having cancer, such as an ovarian cancer or ovarian serous carcinoma. In some embodiments, acquiring knowledge comprises detecting the presence or absence of a MTAP deletion and/or determining the CCNE1-amp status using any suitable method known in the art or described herein. In some embodiments, knowledge of the presence or absence of a MTAP deletion and the CCNE1-amp status of a sample obtained from an individual having cancer, e.g., an ovarian cancer or ovarian serous carcinoma, identifies the individual as one who may benefit from an anti-cancer therapy, e.g., an anti-cancer therapy provided herein. In some embodiments, responsive to the knowledge of the presence or absence of a MTAP deletion and the knowledge of the CCNE1-amp status in a sample obtained from an individual having cancer, e.g., an ovarian cancer or ovarian serous carcinoma, the individual is classified as a candidate to receive an anti-cancer therapy, such as an anti-cancer therapy provided herein. In some embodiments, responsive to the knowledge of the presence or absence of a MTAP deletion and the knowledge of the CCNE1-amp status in a sample obtained from an individual having cancer, e.g., an ovarian cancer or ovarian serous carcinoma, the individual is classified or identified as likely to respond to an anti-cancer therapy, such as an anti-cancer therapy provided herein. In some embodiments, the sample is a sample described herein. In some embodiments, the sample comprises cells from the cancer (e.g., ovarian cancer) or is obtained from cells from the cancer (e.g., ovarian cancer). In some embodiments, the methods further comprise generating a report, e.g., as described herein. In some embodiments, the report comprises a treatment, a therapy, or one or more treatment options identified or selected for the individual, e.g., based at least in part on the knowledge of the presence or absence of a MTAP deletion and the knowledge of the CCNE1-amp status in the sample.

Also provided herein are methods of identifying or selecting a treatment, a therapy, or one or more treatment options for an individual having a cancer, such as an ovarian cancer described herein. In some embodiments, the methods comprise obtaining knowledge of the presence of a MTAP deletion and the CCNE1-amp negative status in a sample from the individual having cancer, such as an ovarian cancer or ovarian serous carcinoma. In some embodiments, acquiring knowledge comprises detecting the presence of a MTAP deletion and/or determining the CCNE1-amp negative status using any suitable method known in the art or described herein. In some embodiments, knowledge of the presence of a MTAP deletion and the CCNE1-amp negative status of a sample obtained from an individual having cancer, e.g., an ovarian cancer or ovarian serous carcinoma, identifies the individual as one who may benefit from treatment with an epigenetic modifier (e.g., a PRMT5 inhibitor). In some embodiments, responsive to the knowledge of the presence of a MTAP deletion and the knowledge of the CCNE1-amp negative status in a sample obtained from an individual having cancer, e.g., an ovarian cancer or ovarian serous carcinoma, the individual is classified as a candidate to receive treatment comprising an epigenetic modifier (e.g., a PRMT5 inhibitor). In some embodiments, responsive to the knowledge of the presence of a MTAP deletion and the knowledge of the CCNE1-amp negative status in a sample obtained from an individual having cancer, e.g., an ovarian cancer or ovarian serous carcinoma, the individual is classified or identified as likely to respond to a treatment comprising an epigenetic modifier (e.g., a PRMT5 inhibitor). In some embodiments, the sample is a sample described herein. In some embodiments, the sample comprises cells from the cancer (e.g., ovarian cancer) or is obtained from cells from the cancer (e.g., ovarian cancer). In some embodiments, the methods further comprise generating a report, e.g., as described herein. In some embodiments, the report comprises a treatment, a therapy, or one or more treatment options identified or selected for the individual, e.g., based at least in part on the knowledge of the presence of a MTAP deletion and the knowledge of the CCNE1-amp negative status in the sample. In some aspects, the one or more treatment options comprise an epigenetic modifier (e.g., a PRMT5 inhibitor).

Also provided herein are methods of identifying or selecting a treatment, a therapy, or one or more treatment options for an individual having a cancer, such as an ovarian cancer described herein. In some embodiments, the methods comprise obtaining knowledge of the presence of a MTAP deletion and the CCNE1-amp positive status in a sample from the individual having cancer, such as an ovarian cancer or ovarian serous carcinoma. In some embodiments, acquiring knowledge comprises detecting the presence of a MTAP deletion and/or determining the CCNE1-amp positive status using any suitable method known in the art or described herein. In some embodiments, knowledge of the presence of a MTAP deletion and the CCNE1-amp positive status of a sample obtained from an individual having cancer, e.g., an ovarian cancer or ovarian serous carcinoma, identifies the individual as one who may benefit from a combination treatment comprising an epigenetic modifier (e.g., a PRMT5 inhibitor). In some embodiments, responsive to the knowledge of the presence of a MTAP deletion and the knowledge of the CCNE1-amp positive status in a sample obtained from an individual having cancer, e.g., an ovarian cancer or ovarian serous carcinoma, the individual is classified as a candidate to receive a combination treatment comprising an epigenetic modifier (e.g., a PRMT5 inhibitor). In some embodiments, responsive to the knowledge of the presence of a MTAP deletion and the knowledge of the CCNE1-amp positive status in a sample obtained from an individual having cancer, e.g., an ovarian cancer or ovarian serous carcinoma, the individual is classified or identified as likely to respond to a combination treatment comprising an epigenetic modifier (e.g., a PRMT5 inhibitor). In some embodiments, the sample is a sample described herein. In some embodiments, the sample comprises cells from the cancer (e.g., ovarian cancer) or is obtained from cells from the cancer (e.g., ovarian cancer). In some embodiments, the methods further comprise generating a report, e.g., as described herein. In some embodiments, the report comprises a treatment, a therapy, or one or more treatment options identified or selected for the individual, e.g., based at least in part on the knowledge of the presence of a MTAP deletion and the knowledge of the CCNE1-amp positive status in the sample. In some aspects, the one or more treatment options comprise a combination therapy comprising an epigenetic modifier (e.g., a PRMT5 inhibitor). In some aspects, the combination treatment further comprises a Wee1 inhibitor, a CDK2 inhibitor, or a PKMYT1 inhibitor.

Also provided herein are methods of identifying or selecting a treatment, a therapy, or one or more treatment options for an individual having a cancer, such as an ovarian cancer described herein. In some embodiments, the methods comprise obtaining knowledge of the absence of a MTAP deletion and the CCNE1-amp negative status in a sample from the individual having cancer, such as an ovarian cancer or ovarian serous carcinoma. In some embodiments, acquiring knowledge comprises detecting the absence of a MTAP deletion and/or determining the CCNE1-amp negative status using any suitable method known in the art or described herein. In some embodiments, knowledge of the absence of a MTAP deletion and the CCNE1-amp negative status of a sample obtained from an individual having cancer, e.g., an ovarian cancer or ovarian serous carcinoma, identifies the individual as one who may benefit a treatment other than a treatment comprising an epigenetic modifier (e.g., a PRMT5 inhibitor) or a Wee1 inhibitor, a CDK2 inhibitor, or a PKMYT1 inhibitor. In some embodiments, responsive to the knowledge of the absence of a MTAP deletion and the knowledge of the CCNE1-amp negative status in a sample obtained from an individual having cancer, e.g., an ovarian cancer or ovarian serous carcinoma, the individual is classified as a candidate to receive treatment other than a treatment comprising an epigenetic modifier (e.g., a PRMT5 inhibitor) or a Wee1 inhibitor, a CDK2 inhibitor, or a PKMYT1 inhibitor. In some embodiments, responsive to the knowledge of the absence of a MTAP deletion and the knowledge of the CCNE1-amp negative status in a sample obtained from an individual having cancer, e.g., an ovarian cancer or ovarian serous carcinoma, the individual is classified or identified as likely to respond to a treatment other than a treatment comprising an epigenetic modifier (e.g., a PRMT5 inhibitor) or a Wee1 inhibitor, a CDK2 inhibitor, or a PKMYT1 inhibitor. In some embodiments, the sample comprises cells from the cancer (e.g., ovarian cancer) or is obtained from cells from the cancer (e.g., ovarian cancer). In some embodiments, the methods further comprise generating a report, e.g., as described herein. In some embodiments, the report comprises a treatment, a therapy, or one or more treatment options identified or selected for the individual, e.g., based at least in part on the knowledge of the presence of a MTAP deletion and the knowledge of the CCNE1-amp negative status in the sample. In some aspects, the one or more treatment options a treatment other than a treatment comprising an epigenetic modifier (e.g., a PRMT5 inhibitor) or a Wee1 inhibitor, a CDK2 inhibitor, or a PKMYT1 inhibitor. In some aspects, the treatment other than a treatment comprising an epigenetic modifier (e.g., a PRMT5 inhibitor) or a Wee1 inhibitor, a CDK2 inhibitor, or a PKMYT1 inhibitor may comprise the standard of care treatment for ovarian cancer and may comprise an anti-cancer therapy as described herein. The anti-cancer therapy may comprise a platinum based chemotherapeutic agent.

Also provided herein are methods of identifying or selecting a treatment, a therapy, or one or more treatment options for an individual having a cancer, such as an ovarian cancer described herein. In some embodiments, the methods comprise obtaining knowledge of the absence of a MTAP deletion and the CCNE1-amp positive status in a sample from the individual having cancer, such as ovarian cancer or ovarian serous carcinoma. In some embodiments, acquiring knowledge comprises detecting the absence of a MTAP deletion and/or determining the CCNE1-amp positive status using any suitable method known in the art or described herein. In some embodiments, knowledge of the absence of a MTAP deletion and the CCNE1-amp positive status of a sample obtained from an individual having cancer, e.g., an ovarian cancer or ovarian serous carcinoma, identifies the individual as one who may not benefit from a treatment comprising an epigenetic modifier (e.g., a PRMT5 inhibitor). In some embodiments, responsive to the knowledge of the absence of a MTAP deletion and the knowledge of the CCNE1-amp positive status in a sample obtained from an individual having cancer, e.g., ovarian cancer or ovarian serous carcinoma, the individual is classified as a candidate to receive a treatment other than a treatment comprising an epigenetic modifier (e.g., a PRMT5 inhibitor). In some embodiments, responsive to the knowledge of the absence of a MTAP deletion and the knowledge of the CCNE1-amp positive status in a sample obtained from an individual having cancer, e.g., ovarian cancer or ovarian serous carcinoma, the individual is classified or identified as unlikely to respond to a treatment comprising an epigenetic modifier (e.g., a PRMT5 inhibitor). In some embodiments, the sample is a sample described herein. In some embodiments, the sample comprises cells from the cancer (e.g., ovarian cancer) or is obtained from cells from the cancer (e.g., ovarian cancer). In some embodiments, the methods further comprise generating a report, e.g., as described herein. In some embodiments, the report comprises a treatment, a therapy, or one or more treatment options identified or selected for the individual, e.g., based at least in part on the knowledge of the absence of a MTAP deletion and the knowledge of the CCNE1-amp positive status in the sample. In some aspects, the one or more treatment options comprise a treatment other than an epigenetic modifier (e.g., a PRMT5 inhibitor). In some aspects, the treatment other than an epigenetic modifier (e.g., a PRMT5 inhibitor) may be a Wee1 inhibitor, a CDK2 inhibitor, or a PKMYT1 inhibitor. In some aspects, the individual may benefit from treatment comprising a Wee1 inhibitor, a CDK2 inhibitor, or a PKMYT1 inhibitor.

### Methods of Treatment

Also provided herein are methods of treating or delaying progression of a cancer in an individual, such as a cancer provided herein. In some embodiments, the methods of treating or delaying progression of an cancer of the disclosure in an individual, comprising acquiring knowledge of the presence or absence of a MTAP deletion in a sample from the individual and administering to the individual a therapeutically effective amount of an anti-cancer therapy, such as an anti-cancer therapy provided herein. In some embodiments, the methods of treating or delaying progression of a cancer of the disclosure in an individual, comprise administering to the individual an effective amount of an anti-cancer therapy, such as an anti-cancer therapy provided herein, responsive to knowledge of the presence or absence of a MTAP deletion in a sample obtained from the individual. In some embodiments, acquiring knowledge comprises detecting the presence or absence of a MTAP deletion using any suitable method known in the art or described herein. In some embodiments, the sample is a sample described herein. In some embodiments, the sample comprises cells from the cancer or is obtained from cells from the cancer. The methods of treatment disclosed herein may include any of the anti-cancer therapies and/or therapeutic agents described herein, e.g., infra. In some embodiments, the anti-cancer therapy comprises an epigenetic modifier. In some embodiments, the epigenetic modifier is a PRMT inhibitor or a PRMT5 inhibitor.

Also provided herein are methods of treating or delaying progression of a cancer in an individual, such as a cancer provided herein. In some embodiments, the methods of treating or delaying progression of an cancer (e.g., ovarian cancer) of the disclosure in an individual, comprising acquiring knowledge of the presence or absence of a MTAP deletion and acquiring knowledge of a CCNE1-amp status in a sample from the individual and administering to the individual a therapeutically effective amount of an anti-cancer therapy, such as an anti-cancer therapy provided herein. In some embodiments, the methods of treating or delaying progression of a cancer of the disclosure in an individual, comprise administering to the individual an effective amount of an anti-cancer therapy, such as an anti-cancer therapy provided herein, responsive to knowledge of the presence or absence of a MTAP deletion and knowledge of a CCNE1-amp status a sample obtained from the individual. In some embodiments, acquiring knowledge comprises detecting the presence or absence of a MTAP deletion and/or determining the CCNE1-amp status using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer). The methods of treatment disclosed herein may include any of the anti-cancer therapies and/or therapeutic agents described herein, e.g., infra.

Also provided herein are methods of treating or delaying progression of a cancer in an individual, such as a cancer provided herein. In some embodiments, the methods of treating or delaying progression of an cancer (e.g., ovarian cancer) of the disclosure in an individual, comprising acquiring knowledge of the presence of a MTAP deletion and acquiring knowledge of a CCNE1-amp negative status in a sample from the individual and administering to the individual a therapeutically effective amount of an epigenetic modifier (e.g., a PRMT5 inhibitor). In some embodiments, the methods of treating or delaying progression of a cancer of the disclosure in an individual, comprise administering to the individual an effective amount of the epigenetic modifier (e.g., the PRMT5 inhibitor), responsive to knowledge of the presence of a MTAP deletion and knowledge of a CCNE1-amp negative status a sample obtained from the individual. In some embodiments, acquiring knowledge comprises detecting the presence of a MTAP deletion and/or determining the CCNE1-amp negative status using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer). The methods of treatment disclosed herein may include an epigenetic modifier (e.g., a PRMT5 inhibitor). In some embodiments, the epigenetic modifier is a PRMT inhibitor or a PRMT5 inhibitor.

Also provided herein are methods of treating or delaying progression of a cancer in an individual, such as a cancer provided herein. In some embodiments, the methods of treating or delaying progression of an cancer (e.g., ovarian cancer) of the disclosure in an individual, comprising acquiring knowledge of the presence of a MTAP deletion and acquiring knowledge of a CCNE1-amp positive status in a sample from the individual and administering to the individual a therapeutically effective amount of combination therapy comprising an epigenetic modifier (e.g., a PRMT5 inhibitor). In some embodiments, the methods of treating or delaying progression of a cancer of the disclosure in an individual, comprise administering to the individual an effective amount of a combination treatment comprising an epigenetic modifier (e.g., a PRMT5 inhibitor), responsive to knowledge of the presence of a MTAP deletion and knowledge of a CCNE1-amp positive status a sample obtained from the individual. In some embodiments, acquiring knowledge comprises detecting the presence of a MTAP deletion and/or determining the CCNE1-amp positive status using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer). The methods of treatment disclosed herein may include a combination treatment comprising an epigenetic modifier (e.g., a PRMT5 inhibitor). In some aspects, the combination treatment further comprises a Wee1 inhibitor, a CDK2 inhibitor, or a PKMYT1 inhibitor.

Also provided herein are methods of treating or delaying progression of a cancer in an individual, such as a cancer provided herein. In some embodiments, the methods of treating or delaying progression of an cancer (e.g., ovarian cancer) of the disclosure in an individual, comprising acquiring knowledge of the absence of a MTAP deletion and acquiring knowledge of a CCNE1-amp negative status in a sample from the individual and administering to the individual a therapeutically effective amount of a treatment other than a treatment comprising an epigenetic modifier (e.g., a PRMT5 inhibitor) or a Wee1 inhibitor, a CDK2 inhibitor, or a PKMYT1 inhibitor. In some embodiments, the methods of treating or delaying progression of a cancer of the disclosure in an individual, comprise administering to the individual an effective amount of a treatment other than a treatment comprising an epigenetic modifier (e.g., a PRMT5 inhibitor) or a Wee1 inhibitor, a CDK2 inhibitor, or a PKMYT1 inhibitor, responsive to knowledge of the absence of a MTAP deletion and knowledge of a CCNE1-amp negative status a sample obtained from the individual. In some embodiments, acquiring knowledge comprises detecting the absence of a MTAP deletion and/or determining the CCNE1-amp negative status using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer). The methods of treatment disclosed herein may include a treatment other than a treatment comprising an epigenetic modifier (e.g., a PRMT5 inhibitor) or a Wee1 inhibitor, a CDK2 inhibitor, or a PKMYT1 inhibitor. The treatment may comprise the standard of care treatment for cancer (e.g., ovarian cancer) and may comprise an anti-cancer therapy as described herein. The anti-cancer therapy may comprise a platinum based chemotherapeutic agent.

Also provided herein are methods of treating or delaying progression of a cancer in an individual, such as a cancer provided herein. In some embodiments, the methods of treating or delaying progression of an cancer (e.g., ovarian cancer) of the disclosure in an individual, comprising acquiring knowledge of the absence of a MTAP deletion and acquiring knowledge of a CCNE1-amp positive status in a sample from the individual and administering to the individual a therapeutically effective amount of treatment other than a treatment comprising an epigenetic modifier (e.g., a PRMT5 inhibitor). In some embodiments, the methods of treating or delaying progression of a cancer of the disclosure in an individual, comprise administering to the individual an effective amount of a treatment other than a treatment comprising an epigenetic modifier (e.g., a PRMT5 inhibitor), responsive to knowledge of the absence of a MTAP deletion and knowledge of a CCNE1-amp positive status a sample obtained from the individual. In some embodiments, acquiring knowledge comprises detecting the absence of a MTAP deletion and/or determining the CCNE1-amp positive status using any suitable method known in the art or described herein. In some embodiments, the sample is a sample described herein. In some embodiments, the sample comprises cells from the cancer (e.g., ovarian cancer) or is obtained from cells from the cancer (e.g., ovarian cancer). The methods of treatment disclosed herein may include a treatment other than a treatment comprising an epigenetic modifier (e.g., a PRMT5 inhibitor). In some aspects, the treatment comprises a Wee1 inhibitor, a CDK2 inhibitor, or a PKMYT1 inhibitor.

### Reporting

In some embodiments, the methods provided herein comprise generating a report, and/or providing a report to party.

In some embodiments, a report according to the present disclosure comprises information about the presence or absence of a MTAP deletion in a sample from an individual; cancer of the disclosure; or a treatment, a therapy, or one or more treatment options for an individual having cancer.

In some embodiments, a report according to the present disclosure comprises information about the presence or absence of a MTAP deletion and/or a CCNE1-amp status in a sample from an individual; cancer (e.g., ovarian cancer) of the disclosure; or a treatment, a therapy, or one or more treatment options for an individual having cancer (e.g., ovarian cancer).

In some embodiments, a report according to the present disclosure comprises information about the presence or absence of a MTAP deletion in a sample obtained from an individual, such as an individual having cancer, e.g., an ovarian cancer or ovarian serous carcinoma. In some embodiments, a report according to the present disclosure comprises information about a CCNE1-amp status in a sample obtained from an individual, such as an individual having cancer, e.g., an ovarian cancer or ovarian serous carcinoma.

In one embodiment, a report according to the present disclosure indicates that a MTAP deletion has been detected in a sample from the individual and/or the sample has been classified as CCNE1-amp negative. In one embodiment, a report according to the present disclosure indicates that a MTAP deletion has not been detected in a sample from the individual and/or the sample has been classified as CCNE1-amp positive. In one embodiment, a report according to the present disclosure indicates that a MTAP deletion has been detected in a sample from the individual and/or the sample has been classified as CCNE1-amp positive. In one embodiment, a report according to the present disclosure indicates that a MTAP deletion has not been detected in a sample from the individual and/or the sample has been classified as CCNE1-amp negative. In some embodiments, the report comprises an identifier for the individual from which the sample was obtained.

In some embodiments, the report includes information on the role of the presence or absence of an MTAP deletion and/or CCNE1-amp status, in disease, such as in a cancer (e.g., ovarian cancer). Such information can include one or more of: information on prognosis of an cancer, such as a cancer (e.g., ovarian cancer) provided herein; information on resistance of an cancer, such as a cancer (e.g., ovarian cancer) provided herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having an cancer (e.g., ovarian cancer), such as an cancer (e.g. ovarian cancer) identified and characterized in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having an cancer (e.g., ovarian cancer); detecting the presence or absence of a MTAP deletion and classifying the CCNE1-amp status in the sample, or acquiring knowledge of the presence or absence of a MTAP deletion and knowledge of a CCNE1-amp status and generating a report.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having an cancer; detecting the presence or absence of a MTAP deletion, or acquiring knowledge of the presence or absence of a MTAP deletion and generating a report.

In some embodiments, a report generated according to the methods provided herein comprises one or more of: information about the presence or absence of a MTAP deletion in the sample; an identifier for the individual from which the sample was obtained; information on the role of a MTAP deletion, in disease (e.g., such as in ovarian cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report. In some embodiments, the anti-cancer therapy comprises an epigenetic modifier. In some embodiments, the epigenetic modifier is a PRMT inhibitor or a PRMT5 inhibitor.

In some embodiments, a report generated according to the methods provided herein comprises one or more of: information about the CCNE1-amp status in the sample; an identifier for the individual from which the sample was obtained; information on the role of CCNE1-amp status, in disease (e.g., such as in ovarian cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

In some embodiments, a report generated according to the methods provided herein comprises one or more of: information about the presence or absence of the MTAP deletion and the CCNE1-amp status in the sample; an identifier for the individual from which the sample was obtained; information on the role of a MTAP deletion and CCNE1-amp status, in disease (e.g., such as in ovarian cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence or absence of a MTAP deletion and/or CCNE1-amp status, the report is provided or delivered to an individual or entity within any of about 1 day or more, about 7 days or more, about 14 days or more, about 21 days or more, about 30 days or more, about 45 days or more, or about 60 days or more from detecting the presence or absence of a MTAP deletion and/or characterizing a CCNE1-amp status in a sample obtained from an individual (e.g., an individual having ovarian cancer).

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Cancers

In some embodiments, a cancer of the disclosure is acute lymphoblastic leukemia (“ALL”), acute myeloid leukemia (“AML”), adenocarcinoma, adenocarcinoma of the lung, adrenocortical cancer, adrenocortical carcinoma, anal cancer, appendiceal cancer, B-cell derived leukemia, B-cell derived lymphoma, B-cell lymphoma, bladder cancer, brain cancer, breast cancer (e.g., triple negative breast cancer (TNBC) or non-triple negative breast cancer), cancer of the fallopian tube(s), cancer of the testes, carcinoma, cerebral cancer, cervical cancer, cholangiocarcinoma, choriocarcinoma, chronic myelogenous leukemia, central nervous system (CNS) tumor, CNS cancer, colon cancer, colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, epithelial neoplasm, thymoma, esophageal cancer, Ewing's sarcoma, eye cancer (e.g., uveal melanoma), eyelid cancer, follicular lymphoma (“FL”), gall bladder cancer, gastric cancer, gastrointestinal cancer, glioblastoma, polycythemia vera, glioblastoma multiforme, glioma (e.g., lower grade glioma), gullet cancer, head and neck cancer, a hematological cancer, hepatocellular cancer, hepatocellular carcinoma, Hodgkin's lymphoma (HL), a heavy chain disease, intestinum rectum cancer, renal cancer, kidney cancer (e.g., kidney clear cell cancer, kidney chromophobe cancer, kidney clear cell cancer, kidney papillary cancer), large B-cell lymphoma, large intestine cancer, laryngeal cancer, leucosis, leukemia, liver cancer, lung cancer (e.g., lung adenocarcinoma, or non-small cell lung cancer), lymphoma, mammary gland cancer, melanoma (e.g., metastatic malignant melanoma), Hodgkin's disease, Waldenstrom's macroglobulinemia, Merkel cell carcinoma, mesothelioma, monocytic leukemia, multiple myeloma, myeloma, myogenic sarcoma, nasopharyngeal cancer, neuroblastic-derived CNS tumor (e.g., neuroblastoma (NB)), neuroma, astrocytoma, pilocytic astrocytoma, anaplastic astrocytoma, medulloblastoma, craniopharyngioma, ependymoma, pinealoma, hemangioblastoma, acoustic neuroma, oligodendroglioma, meningioma, vestibular schwannoma, adenoma, metastatic brain tumor, spinal tumor, non-Hodgkin's lymphoma (NHL), oral cancer, oral cavity cancer, osteosarcoma, ovarian cancer, ovarian carcinoma, pancreatic adenocarcinoma, pancreatic cancer, peritoneal cancer, pheochromocytoma, primary mediastinal B-cell lymphoma, primary peritoneal cancer, prostate cancer (e.g., hormone refractory prostate adenocarcinoma), rectal cancer (rectum carcinoma), relapsed or refractory classic Hodgkin's Lymphoma (cHL), salivary gland cancer (e.g., salivary gland tumor), skin cancer, small cell lung cancer, small intestine cancer, soft tissue sarcoma, squamous carcinoma, squamous cell carcinoma (e.g., squamous cell carcinoma of the anogenital region, squamous cell carcinoma of the anus, squamous cell carcinoma of the cervix, squamous cell carcinoma of the esophagus, squamous cell carcinoma of the head and neck (SCHNC), squamous cell carcinoma of the lung, squamous cell carcinoma of the penis, squamous cell carcinoma of the vagina, or squamous cell carcinoma of the vulva), stomach cancer, T-cell derived leukemia, T-cell lymphoma, testicular cancer, testicular tumor, thymic cancer, thyroid cancer (thyroid carcinoma), tongue cancer, tunica conjunctiva cancer, urinary bladder cancer, urothelial cell carcinoma, uterine cancer (e.g., uterine endometrial cancer or uterine sarcoma such as uterine carcinosarcoma), uterine endometrial cancer, uterus cancer, vaginal cancer, vulvar cancer, or Wilms'tumor.

In some embodiments, a cancer of the disclosure is a hematologic cancer (e.g., a hematologic malignancy), such as diffuse large B cell lymphoma (“DLBCL”), Hodgkin's lymphoma (“HL”), Non-Hodgkin's lymphoma (“NHL”), follicular lymphoma (“FL”), acute myeloid leukemia (“AML”), acute lymphoblastic leukemia (“ALL”), multiple myeloma (“MM”), acute lymphoblastic B-cell leukemia, acute lymphoblastic T-cell leukemia, acute myeloblastic leukemia, acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), or hairy cell leukemia. In some embodiments, a hematologic cancer of the disclosure, is an acute or a chronic leukemia, such as a lymphoblastic, myelogenous, lymphocytic, or myelocytic leukemia. In some embodiments, a hematologic cancer of the disclosure, is a lymphoma (e.g., Hodgkin's lymphoma, such as relapsed or refractory classic Hodgkin's Lymphoma (cHL), a non-Hodgkin's lymphoma, a diffuse large B-cell lymphoma, or a precursor T-lymphoblastic lymphoma), a lymphoepithelial carcinoma, or a malignant histiocytosis.

In some embodiments, a cancer of the disclosure is a solid tumor (e.g., a solid malignancy), such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma.

In certain embodiments, a cancer of the disclosure is a cancer of the adrenal glands (such as neuroblastoma), bladder cancer (such as urothelial (transitional cell) carcinoma), brain cancer (such as anaplastic astrocytoma or glioblastoma), bone cancer (such as osteosarcoma), bone marrow cancer (such as B-cell acute leukemia (B-ALL) or multiple myeloma), breast cancer (such as invasive ductal carcinoma), head and neck cancer (such as adenocarcinoma, mucoepidermoid carcinoma, squamous cell carcinoma), lymph node cancer, lung cancer (e.g., mucoepidermoid carcinoma, sarcoma, small cell undifferentiated carcinoma, adenocarcinoma, adenosquamous carcinoma, large cell carcinoma, large cell neuroendocrine carcinoma, non-small cell lung carcinoma, non-small cell lung carcinoma not otherwise specified, or squamous cell carcinoma), female reproductive cancer (e.g., cancer of the fallopian tubes such as fallopian tube serous carcinoma; ovarian cancer, such as epithelial carcinoma, epithelial carcinoma not otherwise specified, high grade serous carcinoma, low grade serous carcinoma, serous carcinoma; and uterine cancer, such as carcinosarcoma, endometrial adenocarcinoma, endometrial adenocarcinoma not otherwise specified, papillary serous endometrial adenocarcinoma, leiomyosarcoma, sarcoma, sarcoma not otherwise specified, or smooth muscle tumor of uncertain malignant potential (STUMP)), gallbladder cancer (such as adenocarcinoma), cancer of the gastroesophageal junction (such as adenocarcinoma), lymph node cancer (such as anaplastic large cell lymphoma, B-cell lymphoma, B-cell lymphoma not otherwise specified, diffuse large B cell lymphoma, non-Hodgkin's lymphoma, non-Hodgkin's lymphoma not otherwise specified), colon cancer (such as adenocarcinoma), colorectal cancer, skin cancer (such as melanoma or squamous cell carcinoma), small intestine cancer (adenocarcinoma), soft tissue cancer (such as Ewing sarcoma, fibrosarcoma, histiocytosis, histiocytosis not otherwise specified, juvenile xanthogranuloma or non-Langerhans cell histiocytosis, inflammatory myofibroblastic tumor, leiomyosarcoma, neurofibroma, neuroblastoma, sarcoma not otherwise specified, sarcoma, undifferentiated sarcoma, or an undifferentiated soft tissue cancer), pancreatic cancer (such as carcinoma, carcinoma not otherwise specified, ductal adenocarcinoma, or mucinous cystadenocarcinoma), prostate cancer (such as acinar adenocarcinoma), pericardium cancer (such as mesothelioma), peritoneum cancer (such as mesothelioma), salivary gland cancer (such as carcinoma or carcinoma not otherwise specified), stomach cancer (such as adenocarcinoma, adenocarcinoma not otherwise specified, or diffuse type cancer), kidney cancer (such as renal cell carcinoma or renal cell carcinoma not otherwise specified), thyroid cancer (such as carcinoma, carcinoma not otherwise specified, or papillary carcinoma), or a cancer of unknown primary origin (such as adenocarcinoma, carcinoma, carcinoma not otherwise specified, leiomyosarcoma, malignant neoplasm, malignant neoplasm not otherwise specified, melanoma, myoepithelial carcinoma, squamous cell carcinoma (SCC), or undifferentiated neuroendocrine carcinoma).

In some embodiments, a cancer of the disclosure, is ovarian cancer. In some aspects, the ovarian cancer may be a serous ovarian cancer, such as an ovarian serous carcinoma. In some aspects, the ovarian cancer is an epithelial ovarian cancer such as a serous, endometrioid, clear cell or mucinous ovarian cancer. In some aspects, the ovarian cancer is a germ cell tumor, such as a dysgerminoma, endodermal sinus tumor or choriocarcinoma. In some instances, the ovarian cancer may develop from the connective tissues and may include a granulosa cell tumor, a Sertoli-Leydig cell tumor, or a Thecoma-Fibroma tumor. In some embodiments, the ovarian cancer of the disclosure, is stage I, stage II, stage III, or stage IV ovarian cancer.

In certain embodiments, a cancer of the disclosure is a female reproductive cancer (e.g., cancer of the fallopian tubes such as fallopian tube serous carcinoma; ovarian cancer, such as epithelial carcinoma, epithelial carcinoma not otherwise specified, high grade serous carcinoma, low grade serous carcinoma, serous carcinoma. In some embodiments, a cancer of the disclosure is a cancer that is recurrent or refractory to one or more prior anti-cancer therapies.

### Samples

The methods provided herein comprise obtaining knowledge about a sample obtained from an individual with cancer, e.g., an ovarian cancer or ovarian serous carcinoma. The samples of the disclosure may comprise a tissue biopsy sample or a liquid biopsy sample.

In some embodiments, the sample is obtained from an individual having ovarian cancer, such as an ovarian cancer described herein. A variety of materials (such as tissues) can be the source of the nucleic acid used in the methods provided herein. For example, the sample can be solid tissue as from a fresh, frozen and/or preserved organ, tissue sample, biopsy, resection, smear, or aspirate; blood or any blood constituents; bodily fluids such as cerebrospinal fluid, amniotic fluid, urine, saliva, sputum, peritoneal fluid or interstitial fluid; or cells from any time in gestation or development of an individual. In some embodiments, the sample is blood or blood constituents. In some embodiments, the sample is a tumor sample. In some embodiments, the sample is or comprises biological tissue or fluid. In some embodiments, the sample can contain compounds that are not naturally intermixed with the tissue in nature, such as preservatives, anticoagulants, buffers, fixatives, nutrients, antibiotics or the like. In some embodiments, the sample comprises genomic or subgenomic DNA fragments, or RNA, such as mRNA isolated from a sample, e.g., a tumor sample, a normal adjacent tissue (NAT) sample, a tissue sample, or a blood sample obtained from an individual. In some embodiments, the sample comprises cDNA derived from an mRNA sample or from a sample comprising mRNA. In some embodiments, the tissue is preserved as a frozen sample or as a formaldehyde- or paraformaldehyde-fixed paraffin-embedded (FFPE) tissue preparation. For example, the sample can be embedded in a matrix, e.g., an FFPE block or a frozen sample.

In some embodiments, the sample comprises cell-free DNA (cfDNA). In some embodiments, the sample comprises cell-free RNA (cfRNA). In some embodiments, the sample comprises circulating tumor DNA (ctDNA).

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example nucleic acids or proteins extracted from a sample or obtained by subjecting a primary sample to techniques such as amplification or reverse transcription of mRNA, or isolation and/or purification of certain components. In some embodiments, the sample can contain compounds that are not naturally intermixed with the tissue in nature, such as preservatives, anticoagulants, buffers, fixatives, nutrients, antibiotics or the like.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor nucleic acids, such as nucleic acids from a tumor or a cancer sample, e.g., genomic DNA, RNA, or cDNA derived from RNA, from a tumor or cancer sample. In certain embodiments, a tumor nucleic acid sample is purified or isolated (e.g., it is removed from its natural state).

In some embodiments, the sample is a control nucleic acid sample or a reference nucleic acid sample, e.g., genomic DNA, RNA, or cDNA derived from RNA, not containing a mutation or gene fusion described herein. In certain embodiments, the reference or control nucleic acid sample comprises a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

In some embodiments, a sample for use according to the methods of the disclosure, is a solid tissue, e.g., from a fresh, frozen and/or preserved organ, tissue sample, biopsy (e.g., a tumor biopsy), resection, smear, or aspirate; blood or any blood constituents; bodily fluids such as cerebrospinal fluid, amniotic fluid, urine, saliva, sputum, peritoneal fluid or interstitial fluid; or cells such as tumor cells. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some embodiments, the source of the sample is blood or blood constituents. In some embodiments, the source of the sample is a tumor sample. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, proteins extracted from a sample or obtained by subjecting a primary sample to techniques such as isolation and/or purification of certain components.

In some embodiments, the sample comprises tumor proteins or polypeptides, such as proteins or polypeptides from a tumor or a cancer sample. In certain embodiments, the proteins are purified or isolated (e.g., removed from their natural state).

### Anti-cancer Therapies and Formulations

Certain aspects of the present disclosure relate to anti-cancer therapies. In some embodiments, an anti-cancer therapy of the disclosure includes one or more therapeutic agents, e.g., for treating a disease, disorder, associated with an cancer (e.g., ovarian cancer), such as an ovarian serous carcinoma. The cancer (e.g., ovarian cancer) may be characterized by the presence or absence of a MTAP deletion and a CCNE1-amp status in a sample from the individual with the cancer (ovarian cancer). The cancer (e.g., ovarian cancer) may be characterized by the presence or absence of a MTAP deletion in a sample from the individual with the cancer.

In some embodiments, the anti-cancer therapy is an epigenetic modifier. In some embodiments, the epigenetic modifier is a PRMT inhibitor. In some embodiments, the epigenetic modifier is a PRMT5 inhibitor. In some embodiments, the epigenetic modifier is a PRMT inhibitor or a PRMT5 inhibitor.

In some aspect, the anti-cancer therapy is a PRMT5 inhibitor. In some aspects, the anti-cancer therapy is a Wee1 inhibitor, a CDK2 inhibitor, or a PKMYT1 inhibitor. In some aspects, the anti-cancer therapy is a combination therapy comprising a PRMT5 inhibitor and a Wee1 inhibitor, a CDK2 inhibitor, or a PKMYT1 inhibitor. In some aspects, the anti-cancer therapy is a platinum based chemotherapeutic agent.

In some embodiments, the anti-cancer therapy is a small molecule inhibitor, an antibody, a cellular therapy (i.e., a cell-based therapy), or a nucleic acid. In some embodiments, the anti-cancer therapy is a chemotherapeutic agent, an anti-hormonal agent, an antimetabolite chemotherapeutic agent, a kinase inhibitor, a peptide, a gene therapy, a vaccine, a platinum-based chemotherapeutic agent, an immunotherapy, an antibody, or a checkpoint inhibitor. In some embodiments, the anti-cancer therapy comprises a second agent, such as a second anti-cancer agent. In some embodiments, the anti-cancer therapy is administered in combination with a second anti-cancer therapy or agent.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Formulations to be used for in vivo administration are sterile. This is readily accomplished by filtration through sterile filtration membranes or other methods known in the art.

In some embodiments, the anti-cancer therapy is administered as a monotherapy. In some embodiments, the anti-cancer therapy is administered in combination with one or more additional anti-cancer therapies or treatments. In some embodiments, the one or more additional anti-cancer therapies or treatments include one or more anti-cancer therapies described herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

Any of the anti-cancer therapies (optionally as monotherapies or in combination with another therapy or treatment) may find use in any of the methods described herein.

### Kits

Also provided herein are kits for detecting the presence or absence of a MTAP deletion and/or determine a CCNE1-amp status, as described herein. In some embodiments, a kit provided herein comprises a reagent (e.g., one or more oligonucleotides, primers, probes or baits of the present disclosure). In some embodiments, the reagent comprises one or more oligonucleotides, primers, probes or baits of the present disclosure capable of hybridizing to a MTAP and/or CCNE1 nucleic acid molecules. In some embodiments, the reagent comprises one or more oligonucleotides, primers, probes or baits of the present disclosure capable of distinguishing a nucleic acids comprising a MTAP deletion or CCNE1 amplification. In some embodiments, the kit is for use according to any method of detecting MTAP and/or CCNE1 nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting MTAP and/or CCNE1nucleic acid molecules of the disclosure, e.g., using one or more oligonucleotides, primers, probes or baits of the present disclosure and for detecting the presence or absence of a MTAP deletion and/or for determine CCNE1 amp-status.

Also provided herein are kits for detecting a MTAP and/or CCNE1 polypeptide of the disclosure for use in detecting the presence or absence of a MTAP deletion and for determine a CCNE1-amp status. In some embodiments, a kit provided herein comprises a reagent (e.g., one or more antibodies of the present disclosure) for detecting a MTAP and/or CCNE1 polypeptides. In some embodiments, the reagent comprises one or more antibodies of the present disclosure capable of binding to a MTAP or CCNE1 polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a MTAP and/or CCNE1 polypeptide of the disclosure, e.g., using one or more antibodies of the present disclosure and for detecting the presence or absence of a MTAP deletion and/or for determine CCNE1 amp-status.

### Exemplary Embodiments

The following exemplary embodiments are representative of some aspects of the invention:

Embodiment 1: A method of identifying an individual having ovarian cancer that may benefit from a treatment with a PRMT5 inhibitor, said method comprising: acquiring knowledge of a presence of a MTAP deletion in a sample obtained from the individual; acquiring knowledge of a CCNE1 amplification (CCNE1-amp) negative status of the sample; and identifying the individual for the treatment with the PRMT5 inhibitor based on the presence of a MTAP deletion in the sample and CCNE1-amp negative status of the sample.

Embodiment 2. A method of selecting a therapy for an individual having ovarian cancer, said method comprising: acquiring knowledge of a presence of a MTAP deletion in a sample obtained from the individual; acquiring knowledge of a CCNE1 amplification (CCNE1-amp) negative status of the sample; and selecting a treatment comprising a PRMT5 inhibitor based on the presence of a MTAP deletion in the sample and CCNE1-amp negative status of the sample.

Embodiment 3. A method of identifying one or more treatment options for an individual having ovarian cancer, said method comprising: acquiring knowledge of a presence of a MTAP deletion in a sample obtained from the individual; acquiring knowledge of a CCNE1 amplification (CCNE1-amp) negative status of the sample; and generating a report comprising one or more treatment options identified for the individual based at least in part on the presence of a MTAP deletion in the sample and CCNE1-amp negative status of the sample, wherein the one or more treatment options comprise a PRMT5 inhibitor.

Embodiment 4. A method of treating an individual having ovarian cancer, said method comprising: acquiring knowledge of a presence of a MTAP deletion in a sample obtained from the individual; acquiring knowledge of a CCNE1 amplification (CCNE1-amp) negative status of the sample; and administering to the individual treatment comprising a PRMT5 inhibitor based on the presence of a MTAP deletion in the sample and CCNE1-amp negative status of the sample.

Embodiment 5. The method of any of embodiments 1-4, where the CCNE 1-amp negative status is based on CCNE1 copy number normalized by a ploidy of the sample.

Embodiment 6. The method of any of claims 1-5, wherein CCNE1-amp negative status samples have a CCNE 1 copy number 0 above or below the ploidy of the sample.

Embodiment 7. A method of identifying an individual having ovarian cancer that may benefit from a combination treatment comprising a PRMT5 inhibitor, said method comprising: acquiring knowledge of a presence of a MTAP deletion in a sample obtained from the individual; acquiring knowledge of a CCNE1 amplification (CCNE1-amp) positive status of the sample; and identifying the individual for the combination treatment comprising the PRMT5 inhibitor based on the presence of a MTAP deletion in the sample and CCNE1-amp positive status of the sample.

Embodiment 8. A method of selecting a therapy for an individual having ovarian cancer, said method comprising: acquiring knowledge of a presence of a MTAP deletion in a sample obtained from the individual; acquiring knowledge of a CCNE1 amplification (CCNE1-amp) positive status of the sample; and selecting a combination treatment comprising a PRMT5 inhibitor based on the presence of a MTAP deletion in the sample and CCNE1-amp positive status of the sample.

Embodiment 9. A method of identifying one or more treatment options for an individual having ovarian cancer, said method comprising: acquiring knowledge of a presence of a MTAP deletion in a sample obtained from the individual; acquiring knowledge of a CCNE1 amplification (CCNE1-amp) positive status of the sample; and generating a report comprising one or more combination treatment options identified for the individual based at least in part on the presence of a MTAP deletion in the sample and CCNE1-amp positive status of the sample, wherein the one or more combination treatment options comprise a PRMT5 inhibitor.

Embodiment 10. A method of treating an individual having ovarian cancer, said method comprising: acquiring knowledge of a presence of a MTAP deletion in a sample obtained from the individual; acquiring knowledge of a CCNE1 amplification (CCNE1-amp) positive status of the sample; and administering to the individual combination treatment comprising a PRMT5 inhibitor based on the presence of a MTAP deletion in the sample and CCNE1-amp positive status of the sample.

Embodiment 11. The method of embodiments 7-10, wherein the combination treatment further comprises a Wee1 inhibitor, a CDK2 inhibitor, or a PKMYT1 inhibitor.

Embodiment 12. The method of any of embodiments 7-11, wherein the CCNE 1-amp positive status is based on CCNE1 copy number compared to a ploidy of the sample.

Embodiment 13. The method of any of claims 7-12, wherein CCNE 1-amp positive status samples have a CCNE1 copy number 1 or more, 2 or more, or 3 or more above the ploidy of the sample.

Embodiment 14. A method of identifying an individual having ovarian cancer that may not benefit from a treatment with a PRMT5 inhibitor or a Wee1 inhibitor, a CDK2 inhibitor, or a PKMYT1 inhibitor, said method comprising: acquiring knowledge of an absence of a MTAP deletion in a sample obtained from the individual; acquiring knowledge of a CCNE1 amplification (CCNE1-amp) negative status of the sample; and identifying the individual as an individual who may not benefit from a PRMT5 inhibitor or a Wee1 inhibitor, a CDK2 inhibitor, or a PKMYT1 inhibitor based on the absence of a MTAP deletion in the sample and CCNE1-amp negative status of the sample.

Embodiment 15. A method of selecting a therapy for an individual having ovarian cancer, said method comprising: acquiring knowledge of an absence of a MTAP deletion in a sample obtained from the individual; acquiring knowledge of a CCNE1 amplification (CCNE1-amp) negative status of the sample; and selecting a treatment other than a treatment comprising a PRMT5 inhibitor or a Wee1 inhibitor, a CDK2 inhibitor, or a PKMYT1 inhibitor based on the absence of a MTAP deletion in the sample and CCNE1-amp negative status of the sample.

Embodiment 16. A method of identifying one or more treatment options for an individual having ovarian cancer, said method comprising: acquiring knowledge of an absence of a MTAP deletion in a sample obtained from the individual; acquiring knowledge of a CCNE1 amplification (CCNE1-amp) negative status of the sample; and generating a report comprising one or more treatment options identified for the individual based at least in part on the absence of a MTAP deletion in the sample and CCNE1-amp negative status of the sample, wherein the one or more treatment options does not comprise a PRMT5 inhibitor or a Wee1 inhibitor, a CDK2 inhibitor, or a PKMYT1 inhibitor.

Embodiment 17. A method of treating an individual having ovarian cancer, said method comprising: acquiring knowledge of an absence of a MTAP deletion in a sample obtained from the individual; acquiring knowledge of a CCNE1 amplification (CCNE1-amp) negative status of the sample; and administering to the individual a treatment other than a treatment comprising a PRMT5 inhibitor or a Wee1 inhibitor, a CDK2 inhibitor, or a PKMYT1 inhibitor based on the absence of a MTAP deletion in the sample and CCNE1-amp negative status of the sample.

Embodiment 18. The method of any of embodiments 14-17, wherein the individual may benefit from a platinum-based chemotherapeutic agent.

Embodiment 19. The method of any of embodiments14-18, where the CCNE1-amp negative status is based on CCNE1 copy number normalized by a ploidy of the sample.

Embodiment 20. The method of any of embodiments 14-19, wherein CCNE1-amp negative status samples have a CCNE 1 copy number 0 above or below the ploidy of the sample.

Embodiment 21. A method of identifying an individual having ovarian cancer that may not benefit from a treatment with a PRMT5 inhibitor, said method comprising: acquiring knowledge of an absence of a MTAP deletion in a sample obtained from the individual: acquiring knowledge of a CCNE1-amp positive status of the sample; and identifying the individual as an individual who may not benefit from the treatment with the PRMT5 inhibitor based on the absence of a MTAP deletion in the sample and CCNE1-amp positive status of the sample.

Embodiment 22. A method of selecting a therapy for an individual having ovarian cancer, said method comprising: acquiring knowledge of an absence of a MTAP deletion in a sample obtained from the individual: acquiring knowledge of a CCNE1-amp positive status of the sample; and selecting a treatment other than a treatment comprising a PRMT5 inhibitor based on the absence of a MTAP deletion in the sample and CCNE1-amp positive status of the sample.

Embodiment 23. A method of identifying one or more treatment options for an individual having ovarian cancer, said method comprising: acquiring knowledge of an absence of a MTAP deletion in a sample obtained from the individual: acquiring knowledge of a CCNE1-amp positive status of the sample; and generating a report comprising one or more treatment options identified for the individual based at least in part on the absence of a MTAP deletion in the sample and CCNE1-amp positive status of the sample, wherein the one or more treatment options does not comprise a PRMT5 inhibitor.

Embodiment 24. A method of treating an individual having ovarian cancer, said method comprising: acquiring knowledge of an absence of a MTAP deletion in a sample obtained from the individual: acquiring knowledge of a CCNE1-amp positive status of the sample; and administering to the individual a treatment other than a treatment comprising a PRMT5 inhibitor based on the absence of a MTAP deletion in the sample and CCNE1-amp positive status of the sample.

Embodiment 25. The method of any of embodiments 21-24, wherein the individual may benefit from a treatment comprising a Wee1 inhibitor, a CDK2 inhibitor, or a PKMYT1 inhibitor.

Embodiment 26. The method of any of embodiments 21-25, wherein the CCNE 1-amp positive status is based on CCNE1 copy number compared to a ploidy of the sample.

Embodiment 27. The method of any of embodiments 21-25, wherein CCNE1-amp positive status samples have a CCNE1 copy number 1 or more, 2 or more, or 3 or more above the ploidy of the sample.

Embodiment 28. The method of any of embodiments 1-27, wherein the ovarian cancer is ovarian serous carcinoma.

Embodiment 29. The method of any of embodiments 1-28, wherein acquiring knowledge of the presence or absence of MTAP deletion comprises detecting the presence or absence of the MTAP deletion in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Embodiment 30. The method of any of embodiments 1-29, wherein the MTAP deletion is a homozygous deletion.

Embodiment 31. The method of any one of embodiments 1-30, wherein the MTAP deletion comprises a homozygous deletion of the full MTAP gene, a homozygous deletion of MTAP exons 1-8, a homozygous deletion of MTAP exons 5-8, a homozygous deletion of MTAP exons 2-8, a homozygous deletion of MTAP exons 6-8, a homozygous deletion of MTAP exons 7-8, a homozygous deletion of MTAP exon 8, a homozygous deletion of MTAP exons 3-8 or a homozygous deletion of MTAP exons 4-8.

Embodiment 32. The method of any of embodiments 1-6, 14-20 and 28-31, wherein acquiring knowledge of the CCNE1-amp negative status comprises determining a CCNE1-amp status in the sample.

Embodiment 33. The method of any of embodiments 7-13 and 21-32, wherein acquiring knowledge of the CCNE1-amp positive status comprises determining a CCNE1-amp status in the sample.

Embodiment 34. The method of embodiments 32 or 33, wherein determining a CCNE1-amp status in the sample comprises calculating a copy number of CCNE1 in the sample and a ploidy of the sample.

Embodiment 35. The method of embodiment 34 wherein the copy number of CCNE1 in the sample and a ploidy of the sample is determined in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Embodiment 36. The method of embodiment 33 or 35, wherein determining a CCNE1-amp negative status of the sample increases a probability of a detecting a presence of a MTAP deletion in the sample.

Embodiment 37. The method of embodiment 34 or 35, wherein determining a CCNE1-amp positive status of the sample increases a probability of detecting an absence of the MTAP deletion in the sample.

Embodiment 38. The method of any of embodiments 1-37, wherein the sample comprises a tissue biopsy sample or a liquid biopsy.

Embodiment 39. The method of embodiment 38, wherein the sample is a tissue biopsy and comprises a tumor biopsy.

Embodiment 40. The method of embodiment 38, wherein the sample is a liquid biopsy sample and comprises blood, serum, plasma, cerebrospinal fluid, sputum, stool, urine, or saliva.

Embodiment 41. The method of any of embodiments 1-40, wherein the sample comprises cells and/or nucleic acids from the ovarian cancer.

Embodiment 42. The method of embodiment 41, wherein the sample comprises mRNA, DNA, circulating tumor DNA (ctDNA), cell-free DNA, cell-free RNA from the ovarian cancer, or any combination thereof.

Embodiment 43. The method of embodiment 40, wherein the sample is a liquid biopsy sample and comprises circulating tumor cells (CTCs).

Embodiment 44. The method of embodiment 40, wherein the sample is a liquid biopsy sample and comprises cell-free DNA (cfDNA), circulating tumor DNA (ctDNA), or any combination thereof.

Embodiment 45. The method of any one of embodiments 1-44, further comprising obtaining the sample from the individual.

Embodiment 46. The method of any one of embodiments 1-37 and 40-45, further comprising obtaining the liquid biopsy sample from the individual.

Embodiment 47. The method of any one of embodiments 1-39 and 45, further comprising obtaining the tissue biopsy sample from the individual.

Embodiment 48. The method of any one of embodiments 29-47, wherein the sequencing comprises a massively parallel sequencing (MPS) technique, whole genome sequencing (WGS), whole exome sequencing, targeted sequencing, direct sequencing, or a Sanger sequencing technique; an optionally wherein the massively parallel sequencing (MPS) technique comprises next-generation sequencing.

Embodiment 49. The method of embodiment 48, wherein the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample.

Embodiment 50. The method of embodiment 49, wherein the one or more bait molecules comprise one or more nucleic acid molecules, each comprising a region that is complementary to a region of a captured nucleic acid molecule.

Embodiment 51. The method of embodiment 50, wherein the one or more bait molecules each comprise a capture moiety.

Embodiment 52. The method of embodiment 51, wherein the capture moiety is biotin.

Embodiment 53. The method of any of embodiments 1-52, wherein the individual is a patient.

Embodiment 54. The method of any of embodiments 1-53, wherein the individual is a human.

Embodiment 55. A method of identifying an individual having cancer that may benefit from a treatment with an epigenetic modifier, said method comprising: acquiring knowledge of a presence of a MTAP deletion in a sample obtained from the individual; and identifying the individual for the treatment with the epigenetic modifier based on the presence of a MTAP deletion in the sample.

Embodiment 56. A method of selecting a therapy for an individual having cancer, said method comprising: acquiring knowledge of a presence of a MTAP deletion in a sample obtained from the individual; and selecting a treatment comprising an epigenetic modifier based on the presence of a MTAP deletion in the sample.

Embodiment 57. A method of identifying one or more treatment options for an individual having cancer, said method comprising: acquiring knowledge of a presence of a MTAP deletion in a sample obtained from the individual; and generating a report comprising one or more treatment options identified for the individual based at least in part on the presence of a MTAP deletion in the sample, wherein the one or more treatment options comprise a epigenetic modifier.

Embodiment 58. A method of treating an individual having cancer, said method comprising: acquiring knowledge of a presence of a MTAP deletion in a sample obtained from the individual; and administering to the individual treatment comprising a epigenetic modifier based on the presence of a MTAP deletion in the.

Embodiment 59. A method of identifying an individual having cancer that may not benefit from a treatment with an epigenetic modifier, said method comprising: acquiring knowledge of an absence of a MTAP deletion in a sample obtained from the individual; and identifying the individual as an individual who may not benefit from the epigenetic modifier based on the absence of a MTAP deletion in the sample.

Embodiment 60. A method of selecting a therapy for an individual having cancer, said method comprising: acquiring knowledge of an absence of a MTAP deletion in a sample obtained from the individual; and selecting a treatment other than a treatment comprising an epigenetic modifier based on the absence of a MTAP deletion in the sample.

Embodiment 61. A method of identifying one or more treatment options for an individual having cancer, said method comprising: acquiring knowledge of an absence of a MTAP deletion in a sample obtained from the individual; and generating a report comprising one or more treatment options identified for the individual based at least in part on the absence of a MTAP deletion in the sample, wherein the one or more treatment options does not comprise an epigenetic modifier.

Embodiment 62. A method of treating an individual having cancer, said method comprising: acquiring knowledge of an absence of a MTAP deletion in a sample obtained from the individual; and administering to the individual a treatment other than a treatment comprising an epigenetic modifier based on the absence of a MTAP deletion in the sample.

Embodiment 63. The method of any of embodiments 59-62, wherein the individual may benefit from a platinum-based chemotherapeutic agent.

Embodiment 64. The method of any of embodiments 55-63, where in the epigenetic modifier is a PRMT inhibitor or a PRMT5 inhibitor.

Embodiment 65. The method of any of embodiments 55-64, wherein the cancer is ovarian cancer.

Embodiment 66. The method of embodiment 64, wherein the ovarian cancer is ovarian serous carcinoma.

Embodiment 67. The method of any of embodiments 55-66, wherein acquiring knowledge of the presence or absence of MTAP deletion comprises detecting the presence or absence of the MTAP deletion in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Embodiment 68. The method of any of embodiments 55-67, wherein the MTAP deletion is a homozygous deletion.

Embodiment 69. The method of any one of embodiments 55-68, wherein the MTAP deletion comprises a homozygous deletion of the full MTAP gene, a homozygous deletion of MTAP exons 1-8, a homozygous deletion of MTAP exons 5-8, a homozygous deletion of MTAP exons 2-8, a homozygous deletion of MTAP exons 6-8, a homozygous deletion of MTAP exons 7-8, a homozygous deletion of MTAP exon 8, a homozygous deletion of MTAP exons 3-8 or a homozygous deletion of MTAP exons 4-8.

Embodiment 70. The method of any of embodiments 55-69, wherein the sample comprises a tissue biopsy sample or a liquid biopsy.

Embodiment 71. The method of embodiment 70, wherein the sample is a tissue biopsy and comprises a tumor biopsy.

Embodiment 72. The method of embodiment 70, wherein the sample is a liquid biopsy sample and comprises blood, serum, plasma, cerebrospinal fluid, sputum, stool, urine, or saliva.

Embodiment 73. The method of any of embodiments 55-72, wherein the sample comprises cells and/or nucleic acids from the ovarian cancer.

Embodiment 74. The method of embodiment 73, wherein the sample comprises mRNA, DNA, circulating tumor DNA (ctDNA), cell-free DNA, cell-free RNA from the ovarian cancer, or any combination thereof.

Embodiment 75. The method of embodiment 74, wherein the sample is a liquid biopsy sample and comprises circulating tumor cells (CTCs).

Embodiment 76. The method of embodiment 74, wherein the sample is a liquid biopsy sample and comprises cell-free DNA (cfDNA), circulating tumor DNA (ctDNA), or any combination thereof.

Embodiment 77. The method of any one of embodiments 55-76, further comprising obtaining the sample from the individual.

Embodiment 78. The method of any one of embodiments 55-69 and 72-77, further comprising obtaining the liquid biopsy sample from the individual.

Embodiment 79. The method of any one of embodiments 55-71 and 77, further comprising obtaining the tissue biopsy sample from the individual.

Embodiment 80. The method of any one of embodiments 67-79, wherein the sequencing comprises a massively parallel sequencing (MPS) technique, whole genome sequencing (WGS), whole exome sequencing, targeted sequencing, direct sequencing, or a Sanger sequencing technique; an optionally wherein the massively parallel sequencing (MPS) technique comprises next-generation sequencing.

Embodiment 81. The method of embodiment 80, wherein the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample.

Embodiment 82. The method of embodiment 81, wherein the one or more bait molecules comprise one or more nucleic acid molecules, each comprising a region that is complementary to a region of a captured nucleic acid molecule.

Embodiment 83. The method of embodiment 82, wherein the one or more bait molecules each comprise a capture moiety.

Embodiment 84. The method of embodiment 83, wherein the capture moiety is biotin.

Embodiment 85. The method of any of embodiments 55-84, wherein the individual is a patient.

Embodiment 86. The method of any of embodiments 55-85, wherein the individual is a human.

Embodiment 87. A compound, or a pharmaceutically acceptable salt thereof, wherein the compound is an epigenetic modifier capable of inhibiting an epigenetic pathway, and wherein the compound exhibits selective anti-proliferative activity in cancer cells comprising an MTAP deletion.

Embodiment 88. A compound, or a pharmaceutically acceptable salt thereof, wherein the compound inhibits an epigenetic enzyme, and wherein the compound is selectively cytotoxic to cancer cells comprising an MTAP deletion.

Embodiment 89. The compound of embodiment 87 or 88, wherein the MTAP deletion is a homozygous MTAP deletion.

Embodiment 90. A compound, or a pharmaceutically acceptable salt thereof, wherein the compound is an epigenetic modifier for use in the treatment of cancer characterized by a homozygous MTAP deletion.

Embodiment 91. A computer-implemented method for generating a report, the method comprising generating, by a data processing system, a report that lists a compound, or a pharmaceutically acceptable salt thereof, wherein the compound is an epigenetic modifier suitable for use in the treatment of cancer characterized by an MTAP deletion.

Embodiment 92. A computer-implemented method for generating a report, the method comprising generating a report that lists one or more cancer treatment options, wherein at least one of the treatment options comprises an epigenetic modifier suitable for treating cancer characterized by an MTAP deletion.

Embodiment 93. A computer-implemented method for generating a report, the method comprising generating a report that includes information indicative of an MTAP deletion, and a listing of a compound or treatment comprising an epigenetic modifier suitable for use in treating cancer associated with the deletion.

Embodiment 94. A computer-implemented method for generating a report, the method comprising generating a report that lists one or more cancer treatment options that exclude epigenetic modifiers, wherein the report is generated for cancer having an MTAP deletion.

Embodiment 95. The computer-implemented method of any of embodiments 91-94, wherein the MTAP deletion is a homozygous MTAP deletion.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Large NGS-based Comprehensive Genomic Profiling (CGP) of CCNE1 Gain and Amplification (amp) as a Potential Predictive Biomarker of Response in Ovarian Cancer

Pan-tumor tissue (N=479,467) and liquid biopsy (N=7,179) samples underwent next generation sequencing. Genome wide copy number (CN) for each sample was estimates both for the overall sample ploidy and for genomic segments. The degree of amplification of a gene was calculated as an amplification ratio (AmpRatio). The AmpRatio was the CN of the gene over the sample ploidy. Amplifications of the CCNE1gene, defined as CN alterations in the CCNE1 gene greater than the median sample CN. A sample was defined as CCNE1-amp positive if the AmpRatio for CCNE1 was equal to or greater than 3.

For the liquid samples, a ctDNA tumor fraction (TF) was quantified using a multiomics algorithm as described in Sweeny et al, Circulating Tumor DNA Assessment for Treatment Monitoring Adds Value to PSA in Metastatic Castration-Resistant Prostate Cancer, 30(18) Clin Cancer Res (2024).

CCNE1 CN gains detected in 139,907(29 %) pan-tumor tissue samples and the samples has a median AmpRatio for CCEN1 of 1.5 (interquartile range (IQR) 1.3-1.9). 15,425 (3.2% of samples) were characterized as CCNE1-amp positive (CCNE1 AmpRatio equal to or greater than 3). The median AmpRatio for the CCNE1-amp positive samples was 5.2 (IQR 3.8-8.9). Low level CCNE1 gains (AmpRatio less than 3) were most common in small cell lung cancer, bladder cancer, and urinary cancer. 39% of small cell lung cancer samples, 38% of bladder cancer samples, and 38% of urinary cancer samples had low level CCNE1 gains. CCNE1-amp positive was most common in ovarian cancer (12% of samples), uterine/endometrial cancer (9.5% of samples), and gallbladder cancer (9.4% of samples) tumors, these cancers had median AmpRatios of 5.1, 5.0 and 11, respectively. In ovarian tumors, CCNE1 amps were enriched in serous carcinomas (14% of samples) and carcinosarcomas (13% of samples), but less common in endometrioid and clear cell carcinomas (2.2% each per cancer samples). The AmpRatio distribution between ovarian subtypes were similar (4.3-5.4).

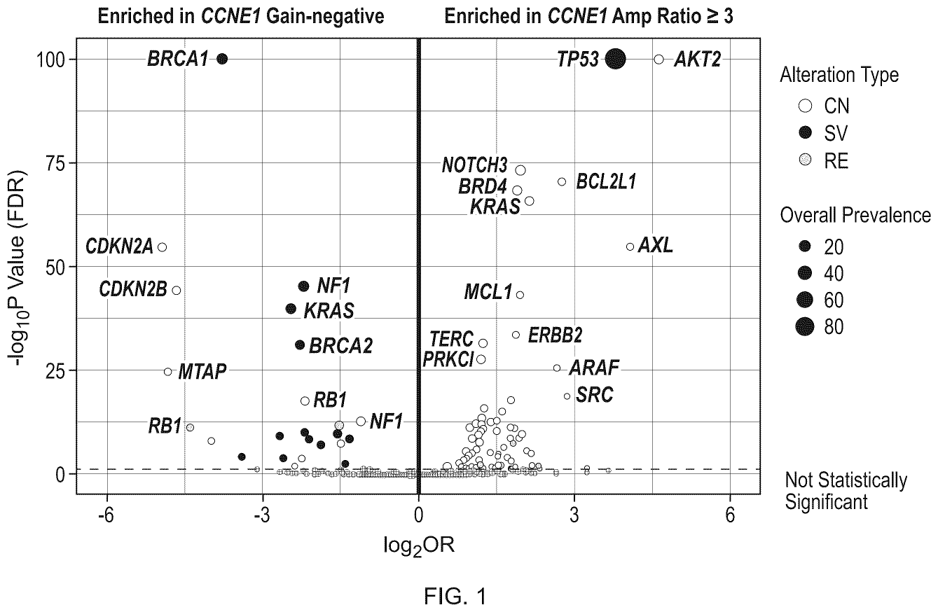

The serous ovarian cancer samples classified as CCNE1-amp positive and the serous ovarian cancer samples with no detected CCNE1 CN gain were compared. As shown in FIG. 1, enrichment of alterations were identified for in each group. After performing FDR correction, MTAP loss was significantly enriched in the CCNE1 gain-negative cohort (p<0.001).

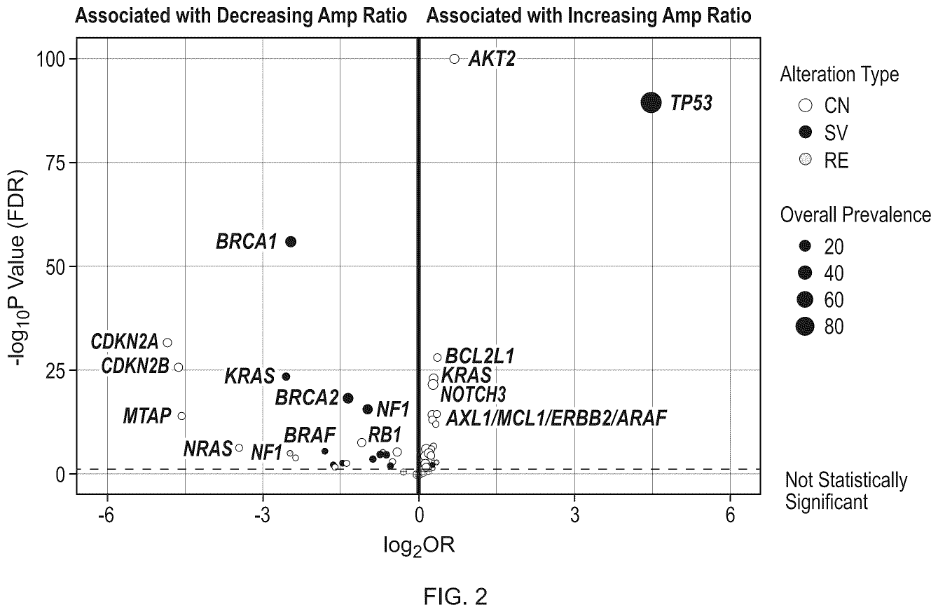

For all the genes with an alteration with a significant (p<0.05) enrichment in either cohort, a logistic regression was performed (FIG. 2). CCNE1-amp ratio as a continuous independent variable, and presence of the gene alteration as the dependent variable. TP53 mutations and AKT2, BCL2L1, KRAS, and NOTCH3 amps were associated with increasing CCNE1 AmpRatio (all p<0.001) while CDKN2A/B/MTAP loss and BRCA1/2, KRAS, NF1, and BRAF mutations were depleted as CCNE1 AmpRatio increased (all p<0.001). Homozygous deletion of MTAP (MTAP loss) was also significantly associated with decreasing CCNE1-amp ratio (p<0.001).

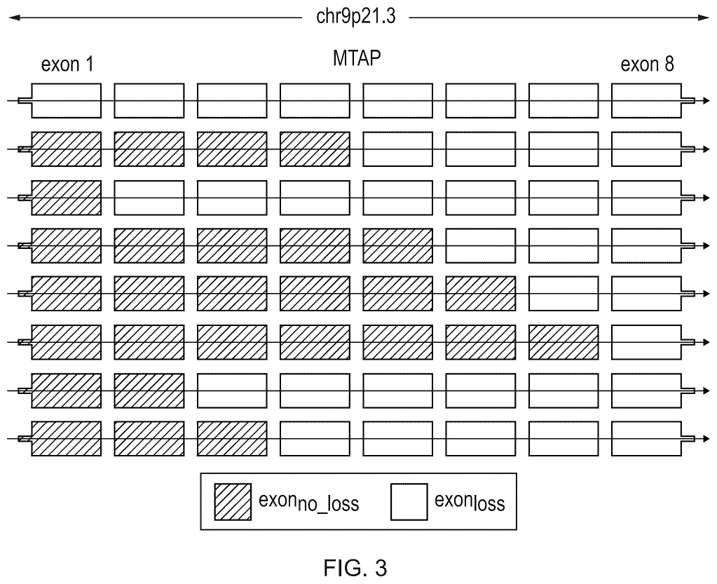

The MTAP loss (homozygous, loss of function) genotypes were reviewed in the serous ovarian cancer samples. FIG. 3 provides a summary of the deleted exons. 82.56% of the samples had deletions of exons 1-8, 4.94% has deletions of exons 5-8, 4.32% had deletions of exons 2-8, 3.99% had deletions of exons 6-8, 1.25% had deletions of exons 7 and 8, 0.9%5% has deletions of exon 8, 0.57% had deletions of exons 3-8, and 0.50% had deletions of exons 4-8.

CCNE1 amps were detected in 1.1% of pan-tumor liquid samples (median TF=23%). In a cohort of 3,185 patients with paired tissue and liquid samples, sensitivity of CCNE1-amp detection in liquid was 49% which increased to 95% in samples with TF≥20%. Detection in liquid correlated with higher AmpRatio and TF.

CCNE1-amp positive statuses were prevalent in ovarian cancers samples with an enrichment in serous and carcinosarcoma subtypes. AmpRatio-dependent genomic differences were observed in serous ovarian samples with varying degrees of CCNE1 gain.

Tumor samples and liquid samples were used to demonstrate the level of tumor shed that allows for reliable detection of amplification status in liquid biopsies. The data provided insight into the relationship between amplification ratio for CCNE1 or genomic alterations, and tumor types which may be vital for ongoing clinical development of CCNE1-targeted therapies.

What is claimed is:

1. A method of identifying an individual having cancer that may benefit from a treatment with an epigenetic modifier, said method comprising:
acquiring knowledge of a presence of a MTAP deletion in a sample obtained from the individual; and
identifying the individual for the treatment with the epigenetic modifier based on the presence of a MTAP deletion in the sample.

2. A method of selecting a therapy for an individual having cancer, said method comprising:
acquiring knowledge of a presence of a MTAP deletion in a sample obtained from the individual; and
selecting a treatment comprising an epigenetic modifier based on the presence of a MTAP deletion in the sample.

3. A method of identifying one or more treatment options for an individual having cancer, said method comprising:
acquiring knowledge of a presence of a MTAP deletion in a sample obtained from the individual; and
generating a report comprising one or more treatment options identified for the individual based at least in part on the presence of a MTAP deletion in the sample, wherein the one or more treatment options comprise a epigenetic modifier.

4. A method of treating an individual having cancer, said method comprising:
acquiring knowledge of a presence of a MTAP deletion in a sample obtained from the individual; and
administering to the individual treatment comprising a epigenetic modifier based on the presence of a MTAP deletion in the sample.

5. The method of claim 1, wherein the epigenetic modifier is a PRMT inhibitor or a PRMT5 inhibitor.

6. The method of claim 1, wherein the cancer is ovarian cancer.

7. The method of claim 6, wherein the ovarian cancer is ovarian serous carcinoma.

8. The method of claim 1, wherein acquiring knowledge of the presence or absence of MTAP deletion comprises detecting the presence or absence of the MTAP deletion in the sample by one or more of: a nucleic acid hybridization assay, an amplification based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

9. The method of claim 1, wherein the MTAP deletion is a homozygous deletion.

10. The method of claim 1, wherein the MTAP deletion comprises a homozygous deletion of the full MTAP gene, a homozygous deletion of MTAP exons 1-8, a homozygous deletion of MTAP exons 5-8, a homozygous deletion of MTAP exons 2-8, a homozygous deletion of MTAP exons 6-8, a homozygous deletion of MTAP exons 7-8, a homozygous deletion of MTAP exon 8, a homozygous deletion of MTAP exons 3-8 or a homozygous deletion of MTAP exons 4-8.

11. The method of claim 1, wherein the sample comprises a tissue biopsy sample or a liquid biopsy.

12. The method of claim 11, wherein the sample is a tissue biopsy and comprises a tumor biopsy.

13. The method of claim 11, wherein the sample is a liquid biopsy sample and comprises blood, serum, plasma, cerebrospinal fluid, sputum, stool, urine, or saliva.

14. The method of claim 1, wherein the sample comprises cells and/or nucleic acids from the cancer.

15. The method of claim 14, wherein the sample comprises mRNA, DNA, circulating tumor DNA (ctDNA), cell-free DNA, cell-free RNA from the cancer, or any combination thereof.

16. The method of claim 15, wherein the sample is a liquid biopsy sample and comprises circulating tumor cells (CTCs).

17. The method of claim 16, wherein the sample is a liquid biopsy sample and comprises cell-free DNA (cfDNA), circulating tumor DNA (ctDNA), or any combination thereof.

18. The method of claim 1, further comprising obtaining the sample from the individual.

19. The method of claim 13, further comprising obtaining the liquid biopsy sample from the individual.

20. The method of claim 11, further comprising obtaining the tissue biopsy sample from the individual.

21. The method of claim 8, wherein the sequencing comprises a massively parallel sequencing (MPS) technique, whole genome sequencing (WGS), whole exome sequencing, targeted sequencing, direct sequencing, or a Sanger sequencing technique; an optionally wherein the massively parallel sequencing (MPS) technique comprises next-generation sequencing.

22. The method of claim 21, wherein the sequencing comprises:
(a) providing a plurality of nucleic acid molecules obtained from the sample, wherein the plurality of nucleic acid molecules comprises a mixture of tumor nucleic acid molecules and non-tumor nucleic acid molecules;
(b) optionally, ligating one or more adapters onto one or more nucleic acid molecules from the plurality of nucleic acid molecules;
(c) amplifying nucleic acid molecules from the plurality of nucleic acid molecules;
(d) optionally, capturing nucleic acid molecules from the amplified nucleic acid molecules, wherein the captured nucleic acid molecules are captured from the amplified nucleic acid molecules by hybridization to one or more bait molecules; and
(e) sequencing, by a sequencer, the captured nucleic acid molecules to obtain a plurality of sequence reads corresponding to one or more genomic loci within a subgenomic interval in the sample.

23. The method of claim 22, wherein the one or more bait molecules comprise one or more nucleic acid molecules, each comprising a region that is complementary to a region of a captured nucleic acid molecule.

24. The method of claim 23, wherein the one or more bait molecules each comprise a capture moiety.

25. The method of claim 24, wherein the capture moiety is biotin.

26. The method of claim 1, wherein the individual is a patient.

27. The method of claim 1, wherein the individual is a human.
